(19) United States
(12) Patent Application Publication
(10) Pub. No.: US 2014/0113972 A1
(43) Pub. Date: Apr. 24, 2014

(54) PRESELECTION OF SUBJECTS FOR THERAPEUTIC TREATMENT WITH ELESCLOMOL BASED ON HYPOXIC STATUS

(71) Applicants: Ronald K. Blackman, Brookline, MA (US); Vojo Vukovic, Winchester, MA (US)

(72) Inventors: Ronald K. Blackman, Brookline, MA (US); Vojo Vukovic, Winchester, MA (US)

(73) Assignee: SYNTA PHARMACEUTICALS CORP., Lexington, MA (US)

(21) Appl. No.: 14/141,883

(22) Filed: Dec. 27, 2013

Related U.S. Application Data
(63) Continuation of application No. 13/300,231, filed on Nov. 18, 2011, now abandoned.
(60) Provisional application No. 61/415,104, filed on Nov. 18, 2010.

(51) Int. Cl. A61K 31/166; G01N 33/574; C12Q 1/68

(52) U.S. Cl. CPC A61K 31/166; C12Q 1/6886; G01N 33/57484
USPC 514/599; 435/26; 435/7.4; 435/34; 435/6.11; 435/7.92; 435/6.12; 435/15; 435/7.23

(57) ABSTRACT

The present invention provides methods for the preselection of a subject for therapeutic treatment with elesclomol based on modulated levels of hypoxia in the subject. In one embodiment, the invention provides methods for the preselection of a subject for therapeutic treatment with elesclomol based on modulated levels of lactate dehydrogenase (LDH). The invention also provides methods for treating cancer in a subject by administering an effective amount of elesclomol to the subject, wherein the subject has a modulated level of hypoxia. The invention further provides kits to practice the methods of the invention.

### CROSS REFERENCE TO RELATED APPLICATIONS

This application claims priority to U.S. Provisional Patent Application Ser. No. 61/415,104 filed on Nov. 18, 2010. The application is incorporated herein by reference in its entirety.

### BACKGROUND OF THE INVENTION

As solid tumors grow, they outgrow their supply of oxygen and nutrients and become hypoxic. Tumor hypoxia is associated with the induction of angiogenesis and has been associated with worse prognosis and poorer treatment response in cancer patients. Hypoxic tumor cells are more invasive and metastatic and also more resistant to killing by radiation treatment and chemotherapy. They are also more genetically unstable and more resistant to apoptosis. Tumor hypoxia upregulates epidermal growth factor receptor (EGFR) expression. (Franovic A et al. PNAS 104: 13092-13097 (2007)). Activation of EGFR on tumor cells leads to phosphorylation of tyrosine residues in the kinase domain of the receptor and activation of signal transduction pathways, including the Ras/Maf/MAPK or PI3K/Akt/mTOR pathways. Activation of these pathways results in nuclear activation of genes related to angiogenesis, cell proliferation, growth, metastasis, and adhesion. (Langer C & Soria J-C. Clinical Lung Cancer, 11:82-90 (2010)). Hypoxia-inducible factors (HIFs) are upregulated in response to hypoxia and also in response to activation of the mammalian target of rapamycin (mTOR) pathway, even under normoxic conditions. (See e.g., Melillo G. Cancer Metastasis Rev 26: 341-352 (2007)). Targets of HIFs include genes involved in glycolytic pathways.

Elesclomol (STA-4783) is disclosed in U.S. Pat. No. 7,795,313 (incorporated herein by reference) a compound represented by the formula N-malonyl-bis(N′-methyl-N′-thiobenzoylhydrazide) shown below

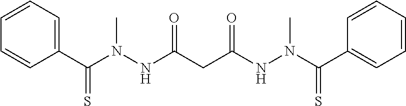 [SMILES: CN(NC(=O)CC(=O)NN(C)C(=S)c1ccccc1)C(=S)c1ccccc1]

Elesclomol triggers apoptosis in cancer cells by targeting mitochondrial energy production in cancer cells. Upon infusion, elesclomol binds copper in plasma, which causes a change in conformation that enables its uptake through membranes and into cells. Copper binds to elesclomol in the Cu(II) oxidative state. Once inside mitochondria, an interaction with the electron transport chain (ETC) results in an electron moving from the ETC to elesclomol, and the copper being reduced from Cu(II) to Cu(I). This process causes a chain reaction of redox reactions, increasing oxidative stress, and growing ETC disruption, ultimately overwhelming mitochondrial protective responses and triggers the mitochondrial apoptosis pathway.

Clinical trials for treatment of metastatic melanoma, prostate cancer, solid tumors, recurrent or persistent ovarian epithelial cancer, fallopian tube cancer, lung cancer, and primary peritoneal cancer with elesclomol have been approved or performed.

### SUMMARY OF THE INVENTION

The instant invention surprisingly demonstrates that low levels of hypoxia in a subject can be used to predict whether a patient will respond to treatment with elesclomol. Specifically, the present invention provides methods for the preselection of a subject for therapeutic treatment with elesclomol based on low levels of hypoxia in cancerous cells in the subject. In one embodiment, the invention provides methods for the preselection of a subject for therapeutic treatment with elesclomol based on low levels of lactate dehydrogenase (LDH) in a cell, e.g., a cancerous cell. The invention also provides methods for treating cancer in a subject by administering an effective amount of elesclomol to the subject, wherein the subject has been selected based on a low level of hypoxia. The invention further provides kits to practice the methods of the invention.

The invention provides compositions for use in methods of treating a subjects having cancer, the composition comprising elesclomol, wherein the cancer comprises a tumor with a low level of hypoxia.

In certain embodiments, the cancer is a solid tumor. In certain embodiments, the cancer is a blood tumor, i.e., not a solid tumor. The type of cancer includes, but is not limited to, one or more of the cancer types such as primary cancer, metastatic cancer, breast cancer, colon cancer, rectal cancer, lung cancer, oropharyngeal cancer, hypopharyngeal cancer, esophageal cancer, stomach cancer, pancreatic cancer, liver cancer, gallbladder cancer, bile duct cancer, small intestine cancer, urinary tract cancer, kidney cancer, bladder cancer, urothelium cancer, female genital tract cancer, cervical cancer, uterine cancer, ovarian cancer, choriocarcinoma, gestational trophoblastic disease, male genital tract cancer, prostate cancer, seminal vesicle cancer, testicular cancer, germ cell tumors, endocrine gland tumors, thyroid cancer, adrenal cancer, pituitary gland cancer, skin cancer, hemangiomas, melanomas, sarcomas arising from bone and soft tissues, Kaposi's sarcoma, brain cancer, nerve cancer, ocular cancer, meningial cancer, astrocytoma, glioma, glioblastoma, retinoblastoma, neuroma, neuroblastoma, Schwannoma, meningioma, solid tumors arising from hematopoietic malignancies, leukemia, Hodgkin's lymphoma, non-Hodgkin's lymphoma, Burkitt's lymphoma, metastatic melanoma, recurrent or persistent ovarian epithelial cancer, fallopian tube cancer, primary peritoneal cancer, epithelial ovarian cancer, primary peritoneal serous cancer, non-small cell lung cancer, gastrointestinal stromal tumors, colorectal cancer, small cell lung cancer, melanoma, glioblastoma multiforme, non-squamous non-small-cell lung cancer, malignant glioma, primary peritoneal serous cancer, metastatic liver cancer, neuroendocrine carcinoma, refractory malignancy, triple negative breast cancer, HER2 amplified breast cancer, squamous cell carcinoma, nasopharageal cancer, oral cancer, biliary tract, hepatocellular carcinoma, squamous cell carcinomas of the head and neck (SCCHN), non-medullary thyroid carcinoma, neurofibromatosis type 1, CNS cancer, liposarcoma, leiomyosarcoma, salivary gland cancer, mucosal melanoma, acral/lentiginous melanoma, paraganglioma; pheochromocytoma, advanced metastatic cancer, solid tumor, squamous cell carcinoma, sarcoma, melanoma, endometrial cancer, head and neck cancer, rhabdomysarcoma, multiple myeloma, gastrointestinal stromal tumor, mantle cell lymphoma, gliosarcoma, bone sarcoma, refractory malignancy, advanced metastatic cancer, solid tumor, metastatic melanoma, prostate cancer, solid tumors, recurrent or persistent ovarian epithelial cancer, fallopian tube cancer, lung cancer, and primary peritoneal cancer.

In certain embodiments, the level of hypoxia in a tumor is determined in a subject sample. In certain embodiments, the subject sample is selected from the group consisting of tumor tissue, blood, urine, stool, lymph, cerebrospinal fluid, circulating tumor cells, bronchial lavage, peritoneal lavage, exudate, effusion, and sputum. In certain embodiments, the tumor tissue is tumor tissue is in the subject or removed from the subject.

In certain embodiments, the level of hypoxia is determined by detecting the activity level or expression level of one or more hypoxia modulated polypeptides. In certain embodiments, the activity level or expression level of the one or more hypoxia modulated polypeptides are down regulated in the sample. In certain embodiments, the level of hypoxia is determined by detecting the activity level or expression level of one or more hypoxia modulated polypeptides or using detection methods selected from the group consisting of detection of activity or expression of at least one isoform or subunit of lactate dehydrogenase (LDH), at least one isoform or subunit of hypoxia inducible factor (HIF), at least one pro-angiogenic form of vascular endothelial growth factor (VEGF), phosphorylated VEGF receptor (pKDR) 1, 2, and 3; neurolipin 1 (NRP-1), pyruvate dehydrokinase (PDH-K), ornithine decarboxylase (ODC), glucose transporter-1 (GLUT-1), glucose transporter-2 (GLUT-2), tumor size, blood flow, EF5 binding, pimonidazole binding, PET scan, and probe detection of hypoxia level.

In certain embodiments, the isoform or subunit of LDH comprises one or more selected from the group consisting of, LDH5, LDH4, LDH3, LDH2, LDH1, LDHA and LDHB; or any combination thereof including total LDH. In certain embodiments, the isoform of HIF comprises one or more selected from the group consisting of HIF-1α, HIF-1β, HIF-2α, and HIF-2β; or any combination thereof including total HIF-1 and/or HIF-2. In certain embodiments, the pro-angiogenic isoform of VEGF is any VEGF-A isoform, or any combination of VEGF-A isoforms including total VEGF-A.

In certain embodiments, detection of a low level of activity or expression of at least one LDH isoform or subunit comprises detection of an LDH activity or expression level of an LDH selected from the group consisting of total LDH, LDH5, LDH4, LDH5 plus LDH4, LDH5 plus LDH4 plus LDH3, and LDHA, wherein the activity level or expression level is 0.8 ULN or less. In certain embodiments, detection of a low level of activity or expression of at least one LDH isoform or subunit comprises detection of an LDH activity or expression level of an LDH selected from the group consisting of total LDH, LDH5, LDH4, LDH5 plus LDH4, LDH5 plus LDH4 plus LDH3, and LDHA, wherein the activity level or expression level is 1.0 ULN or less.

In certain embodiments, detection of a low level of hypoxia comprises detection of a change in a ratio or levels or a change in a ratio of normalized levels of hypoxia modulated polypeptides. In certain embodiments, a low level of hypoxia comprises a ratio or a normalized ratio of 1.0 or less of the ULN, wherein the ratio or normalized ratio is selected from the group consisting of the LDHA to LDHB, LDH5 or LDH4 to LDH1, LDH5 or LDH4 to total LDH, LDH5 and LDH4 to LDH1, LDH5 and LDH4 to total LDH, LDH5, LDH4, and LDH3 to LDH1, and LDH5, LDH4, and LDH3 to total LDH.

In certain embodiments, the subject was previously treated with another chemotherapeutic agent. In certain embodiments, the method further includes identifying a subject as having a high level of hypoxia.

The invention provides methods and use of a level of hypoxia in a tumor for identifying a subject for treatment with elesclomol by determining the level of hypoxia in a tumor from the subject, wherein a low level of hypoxia in the sample indicates the subject is likely to respond to therapy with elesclomol.

In certain embodiments, a subject having a high level of hypoxia in the tumor is not likely to respond to therapy with elesclomol.

In certain embodiments, the cancer is a solid tumor. In certain embodiments, the cancer is a blood tumor, i.e., not a solid tumor. The type of cancer includes, but is not limited to, one or more of the cancer types provided herein.

In certain embodiments, the level of hypoxia in a tumor is determined in a subject sample. In certain embodiments, the subject sample is selected from the group consisting of tumor tissue, blood, urine, stool, lymph, cerebrospinal fluid, circulating tumor cells, bronchial lavage, peritoneal lavage, exudate, effusion, and sputum. In certain embodiments, the tumor tissue is tumor tissue is in the subject or removed from the subject.

In certain embodiments, the level of hypoxia is determined by detecting the activity level or expression level of one or more hypoxia modulated polypeptides. In certain embodiments, the activity level or expression level of the one or more hypoxia modulated polypeptides are down regulated in the sample. In certain embodiments, the level of hypoxia is determined by detecting the activity level or expression level of one or more hypoxia modulated polypeptides or using detection methods selected from the group consisting of detection of activity or expression of at least one isoform or subunit of lactate dehydrogenase (LDH), at least one isoform or subunit of hypoxia inducible factor (HIF), at least one pro-angiogenic form of vascular endothelial growth factor (VEGF), phosphorylated VEGF receptor (pKDR) 1, 2, and 3; neurolipin 1 (NRP-1), pyruvate dehydrokinase (PDH-K), ornithine decarboxylase (ODC), glucose transporter-1 (GLUT-1), glucose transporter-2 (GLUT-2), tumor size, blood flow, EF5 binding, pimonidazole binding, PET scan, and probe detection of hypoxia level.

In certain embodiments, the isoform or subunit of LDH comprises one or more selected from the group consisting of, LDH5, LDH4, LDH3, LDH2, LDH1, LDHA and LDHB; or any combination thereof including total LDH. In certain embodiments, the isoform of HIF comprises one or more selected from the group consisting of HIF-1α, HIF-1β, HIF-2α, and HIF-2β; or any combination thereof including total HIF-1 and/or HIF-2. In certain embodiments, the pro-angiogenic isoform of VEGF is any VEGF-A isoform, or any combination of VEGF-A isoforms including total VEGF-A.

In certain embodiments, detection of a low level of activity or expression of at least one LDH isoform or subunit comprises detection of an LDH activity or expression level of an LDH selected from the group consisting of total LDH, LDH5, LDH4, LDH5 plus LDH4, LDH5 plus LDH4 plus LDH3, and LDHA, wherein the activity level or expression level is 0.8 ULN or less. In certain embodiments, detection of a low level of activity or expression of at least one LDH isoform or subunit comprises detection of an LDH activity or expression level of an LDH selected from the group consisting of total LDH, LDH5, LDH4, LDH5 plus LDH4, LDH5 plus LDH4 plus LDH3, and LDHA, wherein the activity level or expression level is 1.0 ULN or less.

In certain embodiments, detection of a low level of hypoxia comprises detection of a change in a ratio or levels or a change in a ratio of normalized levels of hypoxia modulated polypeptides. In certain embodiments, a low level of hypoxia comprises a ratio or a normalized ratio of 1.0 or less of the ULN, wherein the ratio or normalized ratio is selected from the group consisting of the LDHA to LDHB, LDH5 or LDH4 to LDH1, LDH5 or LDH4 to total LDH, LDH5 and LDH4 to LDH1, LDH5 and LDH4 to total LDH, LDH5, LDH4, and LDH3 to LDH1, and LDH5, LDH4, and LDH3 to total LDH.

In certain embodiments, the subject was previously treated with another chemotherapeutic agent. In certain embodiments, the method further includes identifying a subject as having a high level of hypoxia.

The invention provides tests, methods of testing, and the use of a level of hypoxia for the manufacture of a test to select a therapeutic regimen including elesclomol for the treatment of cancer comprising at least one reagent for determining the level of hypoxia of in a subject sample; wherein the level of hypoxia is used to select the treatment regimen including elesclomol. Reagents for use in such tests can include, but are not limited to at least one agent specifically for detection of a level of hypoxia or determining the level of hypoxia in a subject such as an antibody for detection of the expression level of one or more oxygen sensitive peptides including antibodies specific for a phosphorylation state or otherwise modified state of an oxygen sensitive peptide, a substrate for one or more oxygen sensitive peptides, a nucleic acid for detection of the expression level of one or more oxygen sensitive peptides, and a control sample containing a known amount or concentration of an oxygen sensitive peptide and/or nucleic acid.

In certain embodiments, a low level of hypoxia is indicative that a therapeutic regimen with elesclomol should be selected. In certain embodiments, a high level of hypoxia is indicative that a therapeutic regimen with elesclomol should not be selected.

In certain embodiments, the cancer is a solid tumor. In certain embodiments, the cancer is a blood tumor, i.e., not a solid tumor. The type of cancer includes, but is not limited to, one or more of the cancer types provided herein.

In certain embodiments, the level of hypoxia in a tumor is determined in a subject sample. In certain embodiments, the subject sample is selected from the group consisting of tumor tissue, blood, urine, stool, lymph, cerebrospinal fluid, circulating tumor cells, bronchial lavage, peritoneal lavage, exudate, effusion, and sputum. In certain embodiments, the tumor tissue is tumor tissue is in the subject or removed from the subject.

In certain embodiments, the level of hypoxia is determined by detecting the activity level or expression level of one or more hypoxia modulated polypeptides. In certain embodiments, the activity level or expression level of the one or more hypoxia modulated polypeptides are down regulated in the sample. In certain embodiments, the level of hypoxia is determined by detecting the activity level or expression level of one or more hypoxia modulated polypeptides or using detection methods selected from the group consisting of detection of activity or expression of at least one isoform or subunit of lactate dehydrogenase (LDH), at least one isoform or subunit of hypoxia inducible factor (HIF), at least one pro-angiogenic form of vascular endothelial growth factor (VEGF), phosphorylated VEGF receptor (pKDR) 1, 2, and 3; neurolipin 1 (NRP-1), pyruvate dehydrokinase (PDH-K), ornithine decarboxylase (ODC), glucose transporter-1 (GLUT-1), glucose transporter-2 (GLUT-2), tumor size, blood flow, EF5 binding, pimonidazole binding, PET scan, and probe detection of hypoxia level.

In certain embodiments, the isoform or subunit of LDH comprises one or more selected from the group consisting of, LDH5, LDH4, LDH3, LDH2, LDH1, LDHA and LDHB; or any combination thereof including total LDH. In certain embodiments, the isoform of HIF comprises one or more selected from the group consisting of HIF-1α, HIF-1β, HIF-2α, and HIF-2β; or any combination thereof including total HIF-1 and/or HIF-2. In certain embodiments, the pro-angiogenic isoform of VEGF is any VEGF-A isoform, or any combination of VEGF-A isoforms including total VEGF-A.

In certain embodiments, detection of a low level of activity or expression of at least one LDH isoform or subunit comprises detection of an LDH activity or expression level of an LDH selected from the group consisting of total LDH, LDH5, LDH4, LDH5 plus LDH4, LDH5 plus LDH4 plus LDH3, and LDHA, wherein the activity level or expression level is 0.8 ULN or less. In certain embodiments, detection of a low level of activity or expression of at least one LDH isoform or subunit comprises detection of an LDH activity or expression level of an LDH selected from the group consisting of total LDH, LDH5, LDH4, LDH5 plus LDH4, LDH5 plus LDH4 plus LDH3, and LDHA, wherein the activity level or expression level is 1.0 ULN or less.

In certain embodiments, detection of a low level of hypoxia comprises detection of a change in a ratio or levels or a change in a ratio of normalized levels of hypoxia modulated polypeptides. In certain embodiments, a low level of hypoxia comprises a ratio or a normalized ratio of 1.0 or less of the ULN, wherein the ratio or normalized ratio is selected from the group consisting of the LDHA to LDHB, LDH5 or LDH4 to LDH1, LDH5 or LDH4 to total LDH, LDH5 and LDH4 to LDH1, LDH5 and LDH4 to total LDH, LDH5, LDH4, and LDH3 to LDH1, and LDH5, LDH4, and LDH3 to total LDH.

In certain embodiments, the subject was previously treated with another chemotherapeutic agent. In certain embodiments, the method further includes identifying a subject as having a low level of hypoxia.

The invention provides methods and used of elesclomol for preparation of a medicament for treating a subject having cancer, wherein the subject has a tumor with a low level of hypoxia.

In certain embodiments, the cancer is a solid tumor. In certain embodiments, the cancer is a blood tumor, i.e., not a solid tumor. The type of cancer includes, but is not limited to, one or more of the cancer types provided herein.

In certain embodiments, the level of hypoxia in a tumor is determined in a subject sample. In certain embodiments, the subject sample is selected from the group consisting of tumor tissue, blood, urine, stool, lymph, cerebrospinal fluid, circulating tumor cells, bronchial lavage, peritoneal lavage, exudate, effusion, and sputum. In certain embodiments, the tumor tissue is tumor tissue is in the subject or removed from the subject.

In certain embodiments, the level of hypoxia is determined by detecting the activity level or expression level of one or more hypoxia modulated polypeptides. In certain embodiments, the activity level or expression level of the one or more hypoxia modulated polypeptides are down regulated in the sample. In certain embodiments, the level of hypoxia is determined by detecting the activity level or expression level of one or more hypoxia modulated polypeptides or using detection methods selected from the group consisting of detection of activity or expression of at least one isoform or subunit of lactate dehydrogenase (LDH), at least one isoform or subunit of hypoxia inducible factor (HIF), at least one pro-angiogenic form of vascular endothelial growth factor (VEGF), phosphorylated VEGF receptor (pKDR) 1, 2, and 3; neurolipin 1 (NRP-1), pyruvate dehydrokinase (PDH-K), ornithine decarboxylase (ODC), glucose transporter-1 (GLUT-1), glucose transporter-2 (GLUT-2), tumor size, blood flow, EF5 binding, pimonidazole binding, PET scan, and probe detection of hypoxia level.

In certain embodiments, the isoform or subunit of LDH comprises one or more selected from the group consisting of, LDH5, LDH4, LDH3, LDH2, LDH1, LDHA and LDHB; or any combination thereof including total LDH. In certain embodiments, the isoform of HIF comprises one or more selected from the group consisting of HIF-1α, HIF-1β, HIF-2α, and HIF-2β; or any combination thereof including total HIF-1 and/or HIF-2. In certain embodiments, the pro-angiogenic isoform of VEGF is any VEGF-A isoform, or any combination of VEGF-A isoforms including total VEGF-A.

In certain embodiments, detection of a low level of activity or expression of at least one LDH isoform or subunit comprises detection of an LDH activity or expression level of an LDH selected from the group consisting of total LDH, LDH5, LDH4, LDH5 plus LDH4, LDH5 plus LDH4 plus LDH3, and LDHA, wherein the activity level or expression level is 0.8 ULN or less. In certain embodiments, detection of a low level of activity or expression of at least one LDH isoform or subunit comprises detection of an LDH activity or expression level of an LDH selected from the group consisting of total LDH, LDH5, LDH4, LDH5 plus LDH4, LDH5 plus LDH4 plus LDH3, and LDHA, wherein the activity level or expression level is 1.0 ULN or less.

In certain embodiments, detection of a low level of hypoxia comprises detection of a change in a ratio or levels or a change in a ratio of normalized levels of hypoxia modulated polypeptides. In certain embodiments, a low level of hypoxia comprises a ratio or a normalized ratio of 1.0 or less of the ULN, wherein the ratio or normalized ratio is selected from the group consisting of the LDHA to LDHB, LDH5 or LDH4 to LDH1, LDH5 or LDH4 to total LDH, LDH5 and LDH4 to LDH1, LDH5 and LDH4 to total LDH, LDH5, LDH4, and LDH3 to LDH1, and LDH5, LDH4, and LDH3 to total LDH.

In certain embodiments, the subject was previously treated with another chemotherapeutic agent. In certain embodiments, the method further includes identifying a subject as having a low level of hypoxia.

The invention provides business method for decreasing healthcare costs by determining the level of hypoxia in a biological sample from a tumor obtained from a subject; storing the information on a computer processor; determining if the subject would likely benefit from treatment with elesclomol based on the level of hypoxia; and treating the subject only if the subject will likely benefit from treatment, thereby decreasing healthcare costs.

In certain embodiments, the cancer is a solid tumor. In certain embodiments, the cancer is a blood tumor, i.e., not a solid tumor. The type of cancer includes, but is not limited to, one or more of the cancer types provided herein.

In certain embodiments, the level of hypoxia in a tumor is determined in a subject sample. In certain embodiments, the subject sample is selected from the group consisting of tumor tissue, blood, urine, stool, lymph, cerebrospinal fluid, circulating tumor cells, bronchial lavage, peritoneal lavage, exudate, effusion, and sputum. In certain embodiments, the tumor tissue is tumor tissue is in the subject or removed from the subject.

In certain embodiments, the level of hypoxia is determined by detecting the activity level or expression level of one or more hypoxia modulated polypeptides. In certain embodiments, the activity level or expression level of the one or more hypoxia modulated polypeptides are down regulated in the sample. In certain embodiments, the level of hypoxia is determined by detecting the activity level or expression level of one or more hypoxia modulated polypeptides or using detection methods selected from the group consisting of detection of activity or expression of at least one isoform or subunit of lactate dehydrogenase (LDH), at least one isoform or subunit of hypoxia inducible factor (HIF), at least one pro-angiogenic form of vascular endothelial growth factor (VEGF), phosphorylated VEGF receptor (pKDR) 1, 2, and 3; neurolipin 1 (NRP-1), pyruvate dehydrokinase (PDH-K), ornithine decarboxylase (ODC), glucose transporter-1 (GLUT-1), glucose transporter-2 (GLUT-2), tumor size, blood flow, EF5 binding, pimonidazole binding, PET scan, and probe detection of hypoxia level.

In certain embodiments, the isoform or subunit of LDH comprises one or more selected from the group consisting of, LDH5, LDH4, LDH3, LDH2, LDH1, LDHA and LDHB; or any combination thereof including total LDH. In certain embodiments, the isoform of HIF comprises one or more selected from the group consisting of HIF-1α, HIF-1β, HIF-2α, and HIF-2β; or any combination thereof including total HIF-1 and/or HIF-2. In certain embodiments, the pro-angiogenic isoform of VEGF is any VEGF-A isoform, or any combination of VEGF-A isoforms including total VEGF-A.

In certain embodiments, detection of a low level of activity or expression of at least one LDH isoform or subunit comprises detection of an LDH activity or expression level of an LDH selected from the group consisting of total LDH, LDH5, LDH4, LDH5 plus LDH4, LDH5 plus LDH4 plus LDH3, and LDHA, wherein the activity level or expression level is 0.8 ULN or less. In certain embodiments, detection of a low level of activity or expression of at least one LDH isoform or subunit comprises detection of an LDH activity or expression level of an LDH selected from the group consisting of total LDH, LDH5, LDH4, LDH5 plus LDH4, LDH5 plus LDH4 plus LDH3, and LDHA, wherein the activity level or expression level is 1.0 ULN or less.

In certain embodiments, detection of a low level of hypoxia comprises detection of a change in a ratio or levels or a change in a ratio of normalized levels of hypoxia modulated polypeptides. In certain embodiments, a low level of hypoxia comprises a ratio or a normalized ratio of 1.0 or less of the ULN, wherein the ratio or normalized ratio is selected from the group consisting of the LDHA to LDHB, LDH5 or LDH4 to LDH1, LDH5 or LDH4 to total LDH, LDH5 and LDH4 to LDH1, LDH5 and LDH4 to total LDH, LDH5, LDH4, and LDH3 to LDH1, and LDH5, LDH4, and LDH3 to total LDH.

In certain embodiments, the subject was previously treated with another chemotherapeutic agent. In certain embodiments, the method further includes identifying a subject as having a low level of hypoxia.

The invention further provides kits to practice the methods or uses of diagnosis, treatment, or any other method or use provided herein.

In certain embodiments, a kit includes elesclomol and instruction for administration of elesclomol to a subject having a tumor with a low level of hypoxia.

In certain embodiments, the kit includes at least one reagent specifically for detection of a level of hypoxia and instructions for administering elesclomol to a subject with cancer identified as having a low level of hypoxia. It is understood that not all of the components of the kit need to be in a single package.

Other embodiments are provided infra.

### BRIEF DESCRIPTION OF THE DRAWINGS

FIGS. 1A and B show the activity of LDH5 as a percent of total LDH activity in serum samples from nude mice with (A) HCT116 tumors or (B) 786-O tumors relative to tumor volume. FIGS. 1C and D show the protein levels of LDH5 as a percent of total LDH activity in serum samples from nude mice with (C) HCT116 tumors or (D) 786-O tumors relative to tumor volume.

### DETAILED DESCRIPTION OF THE INVENTION

Research has provided the physician with ever more options for therapeutics for the treatment of cancer. However, despite the availability of the new agents, the ability to match a therapeutic agent to a specific patient based not just on the type of tumor, but the characteristic of the tumor, is lacking. The instant invention provides methods of identifying a subject who will likely respond favorably to treatment with elesclomol by determining the level of hypoxia in a tumor, either by looking directly at markers within the tumor tissue or looking at markers in a peripheral sample from the subject, e.g., a bodily fluid such as blood, serum, plasma, lymph, urine, cerebrospinal fluid, fecal matter, circulating tumor cells, bronchial lavage, peritoneal lavage, exudate, effusion, and sputum for the presence of one or more indicators of the level of hypoxia in the tumor.

In order that the present invention may be more readily understood, certain terms are first defined. In addition, it should be noted that whenever a value or range of values of a parameter are recited, it is intended that values and ranges intermediate to the recited values are also intended to be part of this invention.

### I. DEFINITIONS

The articles “a”, “an” and “the” are used herein to refer to one or to more than one (i.e. to at least one) of the grammatical object of the article unless otherwise clearly indicated by contrast. By way of example, “an element” means one element or more than one element.

The term “including” is used herein to mean, and is used interchangeably with, the phrase “including but not limited to”.

The term “or” is used herein to mean, and is used interchangeably with, the term “and/or,” unless context clearly indicates otherwise.

The term “such as” is used herein to mean, and is used interchangeably, with the phrase “such as but not limited to”.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein can be modified by the term about.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

The recitation of a listing of chemical group(s) in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

As used herein, the term “subject” refers to human and non-human animals, including veterinary subjects. The term “non-human animal” includes all vertebrates, e.g., mammals and non-mammals, such as non-human primates, mice, rabbits, sheep, dog, cat, horse, cow, chickens, amphibians, and reptiles. In a preferred embodiment, the subject is a human and may be referred to as a patient.

As used herein, the terms “treat,” “treating” or “treatment” refer, preferably, to an action to obtain a beneficial or desired clinical result including, but not limited to, alleviation or amelioration of one or more signs or symptoms of a disease or condition, diminishing the extent of disease, stability (i.e., not worsening) state of disease, amelioration or palliation of the disease state, diminishing rate of or time to progression, and remission (whether partial or total), whether detectable or undetectable. “Treatment” can also mean prolonging survival as compared to expected survival in the absence of treatment. Treatment need not be curative.

A “therapeutically effective amount” is that amount sufficient to treat a disease in a subject. A therapeutically effective amount can be administered in one or more administrations.

By “diagnosing” and the like, as used herein, refers to a clinical or other assessment of the condition of a subject based on observation, testing, or circumstances for identifying a subject having a disease, disorder, or condition based on the presence of at least one indicator, such as a sign or symptom of the disease, disorder, or condition. Typically, diagnosing using the method of the invention includes the observation of the subject for multiple indicators of the disease, disorder, or condition in conjunction with the methods provided herein. Diagnostic methods provide an indicator that a disease is or is not present. A single diagnostic test typically does not provide a definitive conclusion regarding the disease state of the subject being tested.

The terms “administer”, “administering” or “administration” include any method of delivery of a pharmaceutical composition or agent into a subject's system or to a particular region in or on a subject. In certain embodiments of the invention, an agent is administered intravenously, intramuscularly, subcutaneously, intradermally, intranasally, orally, transcutaneously, or mucosally. In a preferred embodiment, an agent is administered intravenously. Administering an agent can be performed by a number of people working in concert. Administering an agent includes, for example, prescribing an agent to be administered to a subject and/or providing instructions, directly or through another, to take a specific agent, either by self-delivery, e.g., as by oral delivery, subcutaneous delivery, intravenous delivery through a central line, etc.; or for delivery by a trained professional, e.g., intravenous delivery, intramuscular delivery, intratumoral delivery, etc.

As used herein, the term “survival” refers to the continuation of life of a subject which has been treated for a disease or condition, e.g., cancer. The time of survival can be defined from an arbitrary point such as time of entry into a clinical trial, time from completion or failure or an earlier treatment regimen, time from diagnosis, etc.

As used herein, the term “recur” refers to the re-growth of tumor or cancerous cells in a subject in whom primary treatment for the tumor has been administered. The tumor may recur in the original site or in another part of the body. In one embodiment, a tumor that recurs is of the same type as the original tumor for which the subject was treated. For example, if a subject had an ovarian cancer tumor, was treated and subsequently developed another ovarian cancer tumor, the tumor has recurred. In addition, a cancer can recur in or metastasize to a different organ or tissue than the one where it originally occurred.

As used herein, the terms “identify” or “select” refer to a choice in preference to another. In other words, to identify a subject or select a subject is to perform the active step of picking out that particular subject from a group and confirming the identity of the subject by name or other distinguishing feature. With respect to the instant invention, it is understood that identifying a subject or selecting a subject as having a specific level of hypoxia or a specific level of LDH can include any of a number of acts including, but not limited to, performing a test and observing a result that is indicative of a subject having a specific level of hypoxia; reviewing a test result of a subject and identifying the subject as having a specific level of hypoxia; reviewing documentation on a subject stating that the subject has a specific level of hypoxia and identifying the subject as the one discussed in the documentation by confirming the identity of the subject e.g., by an identification card, hospital bracelet, asking the subject for his/her name and/or other personal information to confirm the subjects identity.

As used herein, the term “benefit” refers to something that is advantageous or good, or an advantage. Similarly, the term “benefiting”, as used herein, refers to something that improves or advantages. For example, a subject will benefit from treatment if they exhibit a decrease in at least one sign or symptom of a disease or condition (e.g., tumor shrinkage, decrease in tumor burden, inhibition or decrease of metastasis, improving quality of life (“QOL”), if there is a delay of time to (tumor) progression (“TTP”), if there is an increase of overall survival (“OS”), etc.), or if there is a slowing or stopping of disease progression (e.g., halting tumor growth or metastasis, or slowing the rate of tumor growth or metastasis). A benefit can also include an improvement in quality of life, or an increase in survival time or progression free survival.

The terms “cancer” or “tumor” are well known in the art and refer to the presence, e.g., in a subject, of cells possessing characteristics typical of cancer-causing cells, such as uncontrolled proliferation, immortality, metastatic potential, rapid growth and proliferation rate, decreased cell death/apoptosis, and certain characteristic morphological features. Cancer cells are often in the form of a solid tumor. However, cancer also includes non-solid tumors, e.g., blood tumors, e.g., leukemia, wherein the cancer cells are derived from bone marrow. As used herein, the term “cancer” includes pre-malignant as well as malignant cancers. Cancers include, but are not limited to, acoustic neuroma, acute leukemia, acute lymphocytic leukemia, acute myelocytic leukemia (monocytic, myeloblastic, adenocarcinoma, angiosarcoma, astrocytoma, myelomonocytic and promyelocytic), acute T-cell leukemia, basal cell carcinoma, bile duct carcinoma, bladder cancer, brain cancer, breast cancer, bronchogenic carcinoma, cervical cancer, chondrosarcoma, chordoma, choriocarcinoma, chronic leukemia, chronic lymphocytic leukemia, chronic myelocytic (granulocytic) leukemia, chronic myleogeneous leukemia, colon cancer, colorectal cancer, craniopharyngioma, cystadenocarcinoma, diffuse large B-cell lymphoma, Burkitt's lymphoma, dysproliferative changes (dysplasias and metaplasias), embryonal carcinoma, endometrial cancer, endotheliosarcoma, ependymoma, epithelial carcinoma, erythroleukemia, esophageal cancer, estrogen-receptor positive breast cancer, essential thrombocythemia, Ewing's tumor, fibrosarcoma, follicular lymphoma, germ cell testicular cancer, glioma, heavy chain disease, hemangioblastoma, hepatoma, hepatocellular cancer, hormone insensitive prostate cancer, leiomyosarcoma, liposarcoma, lung cancer, lymphagioendotheliosarcoma, lymphangiosarcoma, lymphoblastic leukemia, lymphoma (Hodgkin's and non-Hodgkin's), malignancies and hyperproliferative disorders of the bladder, breast, colon, lung, ovaries, pancreas, prostate, skin, and uterus, lymphoid malignancies of T-cell or B-cell origin, leukemia, lymphoma, medullary carcinoma, medulloblastoma, melanoma, meningioma, mesothelioma, multiple myeloma, myelogenous leukemia, myeloma, myxosarcoma, neuroblastoma, non-small cell lung cancer, oligodendroglioma, oral cancer, osteogenic sarcoma, ovarian cancer, pancreatic cancer, papillary adenocarcinomas, papillary carcinoma, pinealoma, polycythemia vera, prostate cancer, rectal cancer, renal cell carcinoma, retinoblastoma, rhabdomyosarcoma, sarcoma, sebaceous gland carcinoma, seminoma, skin cancer, small cell lung carcinoma, solid tumors (carcinomas and sarcomas), small cell lung cancer, stomach cancer, squamous cell carcinoma, synovioma, sweat gland carcinoma, thyroid cancer, Waldenstrom's macroglobulinemia, testicular tumors, uterine cancer and Wilms' tumor. Other cancers include primary cancer, metastatic cancer, oropharyngeal cancer, hypopharyngeal cancer, liver cancer, gallbladder cancer, bile duct cancer, small intestine cancer, urinary tract cancer, kidney cancer, urothelium cancer, female genital tract cancer, uterine cancer, gestational trophoblastic disease, male genital tract cancer, seminal vesicle cancer, testicular cancer, germ cell tumors, endocrine gland tumors, thyroid cancer, adrenal cancer, pituitary gland cancer, hemangioma, sarcoma arising from bone and soft tissues, Kaposi's sarcoma, nerve cancer, ocular cancer, meningial cancer, glioblastomas, neuromas, neuroblastomas, Schwannomas, solid tumors arising from hematopoietic malignancies such as leukemias, metastatic melanoma, recurrent or persistent ovarian epithelial cancer, fallopian tube cancer, primary peritoneal cancer, gastrointestinal stromal tumors, colorectal cancer, gastric cancer, melanoma, glioblastoma multiforme, non-squamous non-small-cell lung cancer, malignant glioma, epithelial ovarian cancer, primary peritoneal serous cancer, metastatic liver cancer, neuroendocrine carcinoma, refractory malignancy, triple negative breast cancer, HER2 amplified breast cancer, nasopharageal cancer, oral cancer, biliary tract, hepatocellular carcinoma, squamous cell carcinomas of the head and neck (SCCHN), non-medullary thyroid carcinoma, recurrent glioblastoma multiforme, neurofibromatosis type 1, CNS cancer, liposarcoma; leiomyosarcoma, salivary gland cancer, mucosal melanoma, acral/lentiginous melanoma, paraganglioma, pheochromocytoma, advanced metastatic cancer, solid tumor, triple negative breast cancer, colorectal cancer, sarcoma, melanoma, renal carcinoma, endometrial cancer, thyroid cancer, rhabdomysarcoma, multiple myeloma, ovarian cancer, glioblastoma, gastrointestinal stromal tumor, mantle cell lymphoma, and refractory malignancy.

“Solid tumor,” as used herein, is understood as any pathogenic tumor that can be palpated or detected using imaging methods as an abnormal growth having three dimensions. A solid tumor is differentiated from a blood tumor such a leukemia. However, cells of a blood tumor are derived from bone marrow, therefore, the tissue producing the cancer cells is a solid tissue that can be hypoxic.

“Tumor tissue” is understood as cells, extracellular matrix, and other naturally occurring components associated with the solid tumor.

As used herein, the term “isolated” refers to a preparation that is substantially free (e.g., 50%, 60%, 70%, 80%, 90% or more, by weight) from other proteins, nucleic acids, or compounds associated with the tissue from which the preparation is obtained.

The term “sample” as used herein refers to a collection of similar fluids, cells, or tissues isolated from a subject. The term “sample” includes any body fluid (e.g., urine, serum, blood fluids, lymph, gynecological fluids, cystic fluid, ascetic fluid, ocular fluids and fluids collected by bronchial lavage and/or peritoneal rinsing), ascites, tissue samples (e.g., tumor samples) or a cell from a subject. Other subject samples include tear drops, serum, cerebrospinal fluid, feces, sputum, and cell extracts. In one embodiment, the sample is removed from the subject. In a particular embodiment, the sample is urine or serum. In another embodiment, the sample does not include ascites or is not an ascites sample. In another embodiment, the sample does not include peritoneal fluid or is not peritoneal fluid. In one embodiment, the sample comprises cells. In another embodiment, the sample does not comprise cells. In certain embodiments, the sample can be the portion of the subject that is imaged (e.g., using a PET scan, a functional imaging method such as MRI to detect blood flow) or tested to determine level of hypoxia (e.g., tumor tissue assayed for level of hypoxia using a probe). Samples are typically removed from the subject prior to analysis, however, tumor samples can be analyzed in the subject, for example, using imaging or other detection methods.

In some embodiments, only a portion of the sample is subjected to an assay for determining the level of hypoxia or the level of the tumor using any method provided herein. In certain embodiments, the level of hypoxia is indicated by the level of an isoform or subunit of lactate dehydrogenase (LDH) or any combination of subunits or isoforms including total LDH, or various portions of the sample are subjected to various assays for determining the level of hypoxia or the level of an isoform or subunit of LDH. Also, in many embodiments, the sample may be pre-treated by physical or chemical means prior to the assay. For example, samples, for example, blood samples, can be subjected to centrifugation, dilution and/or treatment with a solubilizing substance prior to assaying the samples for the level of hypoxia or LDH. Such techniques can serve to enhance the accuracy, reliability, and reproducibility of the assays of the present invention.

The term “control sample,” as used herein, refers to any clinically relevant comparative sample, including, for example, a sample from a healthy subject not afflicted with cancer, a sample from a subject having a less severe or slower progressing cancer than the subject to be assessed, a sample from a subject having some other type of cancer or disease, a sample from a subject prior to treatment, a sample of non-diseased tissue (e.g., non-tumor tissue), a sample from the same origin and close to the tumor site, and the like. A control sample may include a sample derived from one or more subjects. A control sample may also be a sample made at an earlier time point from the subject to be assessed. For example, the control sample could be a sample taken from the subject to be assessed before the onset of the cancer, at an earlier stage of disease, or before the administration of treatment or of a portion of treatment. A control sample can be a purified sample, protein, and/or nucleic acid provided with a kit. Such control samples can be diluted, for example, in a dilution series to allow for quantitative measurement of analytes in test samples. The control sample may also be a sample from an animal model, or from a tissue or cell lines derived from the animal model, of the cancer. The level of hypoxia or LDH in a control sample that consists of a group of measurements may be determined, e.g., based on any appropriate statistical measure, such as, for example, measures of central tendency including average, median, or modal values.

The term “control level” refers to an accepted or pre-determined level of hypoxia or LDH which is used to compare with the level of hypoxia or LDH in a sample derived from a subject. For example, in one embodiment, the control level of hypoxia is based on the level of hypoxia in sample(s) from a subject(s) having slow disease progression. In another embodiment, the control level of hypoxia is based on the level in a sample from a subject(s) having rapid disease progression. In another embodiment, the control level of hypoxia is based on the level of hypoxia in a sample(s) from an unaffected, i.e., non-diseased, subject(s), i.e., a subject who does not have cancer. In yet another embodiment, the control level of hypoxia is based on the level of hypoxia in a sample from a subject(s) prior to the administration of a therapy for cancer. In another embodiment, the control level of hypoxia is based on the level of hypoxia in a sample(s) from a subject(s) having cancer that is not contacted with a test compound. In another embodiment, the control level of hypoxia is based on the level of hypoxia in a sample(s) from a subject(s) not having cancer that is contacted with a test compound. In one embodiment, the control level of hypoxia is based on the level of hypoxia in a sample(s) from an animal model of cancer, a cell, or a cell line derived from the animal model of cancer. In another embodiment, the control level of hypoxia is listed in a chart.

In one embodiment, the control is a standardized control, such as, for example, a control which is predetermined using an average of the levels of hypoxia from a population of subjects having no cancer. In still other embodiments of the invention, a control level of hypoxia is based on the level of hypoxia in a non-cancerous sample(s) derived from the subject having cancer. For example, when a biopsy or other medical procedure reveals the presence of cancer in one portion of the tissue, the control level of hypoxia may be determined using the non-affected portion of the tissue, and this control level may be compared with the level of hypoxia in an affected portion of the tissue. Similarly, when a biopsy or other medical procedure reveals the presence of a cancer in one portion of the tissue, the control level of hypoxia may be determined using the non-affected portion of the tissue, and this control level may be compared with the level of hypoxia in an affected portion of the tissue.

As used herein, the term “obtaining” is understood herein as manufacturing, purchasing, or otherwise coming into possession of.

As used herein, the term “lactate dehydrogenase” refers to an enzyme that interconverts pyruvate and lactate with concomitant interconversion of NADH and NAD+. Under conditions of hypoxia, the reaction favors the conversion of pyruvate to lactate. Under conditions of normoxia, or low levels of hypoxia, the reaction favors the conversion of lactate to pyruvate. Functional lactate dehydrogenase are homo or hetero tetramers composed of M and H protein subunits encoded by the LDHA and LDHB genes respectively: LDH-1 (4H) is the predominant form found, for example, in the heart and red blood cells; LDH-2 (3H1M) is the predominant form found, for example, in the reticuloendothelial system; LDH-3 (2H2M) is the predominant form found, for example, in the lungs; LDH-4 (1H3M) is the predominant form found, for example, in the kidneys, placenta and pancreas; and LDH-5 (4M) is found, for example, in the liver and striated muscle. Typically, multiple forms of LDH are found in these tissues. Lactate dehydrogenase is classified as (EC 1.1.1.27). The specific ratios tested may be tumor-type specific.

As used herein, the terms “hypoxia” and “hypoxic” refer to a condition in which a cancer or a tumor has a low oxygen microenvironment or a less well-oxygenated microenvironment. Hypoxia occurs when tumor growth exceeds new blood vessel formation, and a tumor must undergo genetic and adaptive changes to allow them to survive and proliferate in the hypoxic environment. The development of intratumoral hypoxia is a common sign of solid tumors. When a tumor microenvironment is less well-oxygenated, there is a greater dependency on oxygen-sensitive pathways, including but not limited to HIF1α pathways, VEGF pathways, and mTOR pathways. These pathways facilitate crucial adaptive mechanisms, such as angiogenesis, glycolysis, growth-factor signaling, immortalization, genetic instability, tissue invasion and metastasis, apoptosis, and pH regulation (see, e.g., Harris, Nature Reviews, 2:38-47, 2002). These pathways may also facilitate invasion and metastasis. Accordingly, the treatment of a subject with a cancer or tumor with elesclomol is more effective when the subject has a tumor that exhibits a modulated level of hypoxia, e.g., a low level of hypoxia. As the level of hypoxia in the tumor can be determined by obtaining a sample from a site other than the tumor, as used herein, the subject can be stated to demonstrate a modulated level of hypoxia when it is the tumor present in the subject that demonstrates a modulated level of hypoxia. As used herein it is understood that the subject with a modulated level of hypoxia is typically not suffering from systemic oxygen imbalance or ischemic disease at a site remote from the tumor.

As used herein, the term “level of hypoxia” is understood as the amount of one or more markers indicative of a low oxygen level, or cells having characteristics and/or employing biological pathways characteristic of cells with a low oxygen level, e.g., due to the Warburg effect. Such markers include, but are not limited to, lactate dehydrogenase (LDH), at least one isoform or subunit of hypoxia inducible factor (HIF), at least one pro-angiogenic form of vascular endothelial growth factor (VEGF), phosphorylated VEGF receptor (pKDR) 1, 2, or 3; neurolipin 1 (NRP-1), pyruvate dehydrokinase (PDH-K), GLUT-1, GLUT-2, and ornithine decarboxylase (ODC). Tumor size can also be correlated with a level of hypoxia. A level of hypoxia can also be determined by PET scan. LDH can be one or more isoforms or subunits of LDH such as LDH5, LDH4, LDH3, LDH2, LDH1, LDHM (also known as LDHA) and LDHH (also known as LDHB). In another embodiment, the level of hypoxia is determined by determining the ration of two or more forms of LDH, e.g., the ratio of LDH5:LDH1. In one embodiment, LDH can be a total sample of all LDH isoforms or subunits. “Hypoxia inducible factors” or “HIFs” are transcription factors which respond to changes in available oxygen in a cellular environment. HIF1α is a master regulator of hypoxic gene expression and oxygen homeostasis. HIF can be one or more subunits or isoforms of HIF including HIF-1α, HIF-1β, HIF-2α, and HIF-2β. VEGF can be one or more of the various splice forms of VEGF including pro-angiogenic VEGF-A and antiangiogenic VEGF-B.

As used herein, the term “level of LDH” refers to the amount of LDH present in a sample which can be used to indicate the presence or absence of hypoxia in the tumor in the subject from whom the sample was obtained. LDH enables the conversion of pyruvate to lactate and is a critical component of glycolysis under hypoxic conditions. LDH can be total LDH or one or more isoforms or subunits of LDH such as LDH5, LDH4, LDH3, LDH2, LDH1, LDHM (also known as LDHA) and LDHH (also known as LDHB). A modulated level of LDH can refer to a high level of LDH or a low level of LDH. In one embodiment, a PET scan (which is positive when aerobic glycolysis is active) is an indicator of a high level of LDH. In another embodiment, a PET scan (which is negative when aerobic glycolysis is inactive) is an indicator of a low level of LDH. In one embodiment, a high level of LDH is at least 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3, 4, 5, 6, 7, 8, 9, or 10 times the value of normal level of LDH. In another embodiment, a low level of LDH is 0.9, 0.8, 0.7, 0.6, 0.5, 0.4, 0.3, 0.2, or 0.1 times the value of a normal level of LDH. A normal level of LDH, or any other marker, can be defined as any value within the range of normal, or the upper limit of the normal value, or the lower limit of the normal value. Assays for determining the level of LDH in a sample are well known in the art and provided herein.

In another embodiment, the level of LDH can be understood to be a change in the relative levels of protein or activity of LDH isoforms or the ratio of LDH isoforms to each other or to total LDH. A change of the relative levels of the isoforms can be indicative of the level of hypoxia. For example, an increase in the level of LDHA relative to LDHB can be indicative of an increase in hypoxia. Alternatively, an increase in the level of LDH5 and/or LDH4, either individually or in total, relative to the level of LDH1 or total LDH can be indicative of an increase in hypoxia. The relative levels can be compared to relative levels in an appropriate control sample from normal subjects, e.g., subjects without cancer or ischemic disease. The normal levels can be considered to be a range with an upper level of normal and a lower level of normal. In certain embodiments, a high level of LDH can be understood an increase in the level of LDHA or LDH5 and/or LDH4 relative to the level of LDHB or LDH1, respectively, or to total LDH, of at least 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3, 4, 5, 6, 7, 8, 9, or 10 times the value of normal ratio of LDHA or LDH5 and/or LDH4 relative to the level of LDHB or LDH1, respectively, or to total LDH. In another embodiment, a low level of LDH is 0.9, 0.8, 0.7, 0.6, 0.5, 0.4, 0.3, 0.2, or 0.1 LDHA or LDH5 and/or LDH4 relative to the level of LDHB or LDH1, respectively.

As used herein, a “normalized ratio” is understood as a proportion of two values that have been compared to a standard, either an external (e.g., population control level) or an internal (e.g., level from a normal tissue, level from an earlier time point, level of one or more isoforms) control to allow for comparison of samples between individuals. For example, the ratio of normalized levels of hypoxia modulated polypeptides can be determined by determining a ratio of two normalized levels of two isoforms or subunits of LDH or total LDH by comparing the level of a first isoform or subunit of LDH in the sample relative to a control sample to provide a first normalized level, and the level of a second isoform or subunit of LDH or total LDH relative to a control sample to provide a second normalized level, and calculating a ratio of the first normalized level and the second normalized level to provide a normalized ratio of LDH isoforms or subunits, wherein at least one of the first level and the second level are not total LDH. In certain embodiments, a low level of hypoxia is a normalized ratio of the ULN of LDHA to LDHB of 1.0 or less, or a normalized ratio of the ULN of LDH5 and/or LDH4 to LDH1 or total LDH of 1.0 or less.

Assays for determining the level of LDH in a sample are well known in the art. See, e.g., U.S. Publication Nos. 2010/0178283 and 2008/0213744 and U.S. Pat. Nos. 4,250,255 and 6,242,208, the entire contents of each of which are expressly incorporated herein by reference. Nucleic acid and amino acid sequences of various LDH isoforms are known in the art and available in public databases (e.g., at blast.ncbi.nlm.nih.gov/Blast.cgi).

It is also understood that levels of the various markers can include the level of a post-translationally modified marker, e.g., the total amount of an isoform of HIF may remain the same, but the amount of the hydroxylated version of the HIF may increase. In addition, it is noted that HIF and other hypoxia modulated polypeptides can be upregulated by a number of conditions other than hypoxia, e.g., pH change, changes in levels of O₂. or H₂O₂, etc. Accordingly, although the term “level of expression,” as used herein, is intended to encompass all hypoxia responsive factors, a change in their level of expression may or may not actually directly reflect the amount of oxygen available to the tumor.

Methods to detect the levels of markers of hypoxia are well known in the art. Antibodies against and kits for detection of hypoxia modulated polypeptides can be purchased from a number of commercial sources. Alternatively, using routine methods known in the art (e.g., immunization of animals, phage display, etc.) antibodies against one or more hypoxia modulated polypeptides or subunits or isoforms thereof can be made and characterized. Antibodies can be used for the detection of levels of hypoxia using ELISA, RIA, or other immunoassay methods, preferably automated methods, for the quantitative detection of proteins in samples of bodily fluids or homogenized solid samples. Hypoxia can be detected by enzyme activity assays (e.g., LDH activity, kinase activity) including in gel assays to resolve the activity of various isoforms of proteins. Alternatively, immunohistochemical methods can be used on tumor samples and tissue sections. Antibodies against prodrugs that localize in hypoxic regions (e.g., EF5, pimonidazole, etc.) can also be used to detect hypoxia. Functional imaging measuring bloodflow in the tumor can be used as an indicator of hypoxia in the tissue. Direct measurement of hypoxia can be preformed by inserting a sensor into the tumor. Qualitative scoring methods and scanning methods to detect staining are known in the art. When qualitative scoring methods are used, it is preferred that two independent, blinded technicians, pathologists, or other skilled individuals analyze each sample with specific methods for resolving any significant disagreement in scoring, e.g., a third individual reviews the tissue sample.

Alternatively, nucleic acid-based methods of detection of levels of hypoxia are also well known in the art. Methods of designing primers and probes for quantitative reverse transcription real time (rt)PCR are known in the art. Methods for performing northern blots to detect RNA levels are known in the art. Nucleic acid detection methods can also include fluorescence in situ hybridization (FISH) and in situ PCR. Qualitative scoring methods and scanning methods to detect staining are known in the art. When qualitative scoring methods are used, it is preferred that two independent, blinded technicians, pathologists, or other skilled individuals analyze each sample with specific methods for resolving any significant disagreement in scoring, e.g., a third individual reviews the tissue sample. Assays for determining the level of LDH in a sample are well known in the art. See, e.g., U.S. Publication Nos. 2010/0178283 and 2008/0213744 and U.S. Pat. Nos. 4,250,255 and 6,242,208, the entire contents of each of which are expressly incorporated herein by reference.

“Baseline” refers to the level of hypoxia or the level of LDH upon patient entrance into the study or at the initiation of treatment and is used to distinguish from levels of hypoxia or levels of LDH the patient might have during or after treatment.

“Elevated” or “lower” refers to a patient's value relative to the upper limit of normal (“ULN”) or the lower limit of normal (“LLN”) which are based on historical normal control samples. As the level of the hypoxic marker present in the subject will be a result of the disease, and not a result of treatment, typically not a control, a sample obtained from the patient prior to onset of the disease will not likely be available. Because different labs may have different absolute results, LDH values are presented relative to that lab's upper limit of normal value (ULN). LDH can be expressed in IU/ml (International Units per milliliter). An accepted ULN for LDH is 234 IU/ml, however, this value is not universally accepted or applicable to all methods of detection of LDH in all samples.

The specific value for ULN and LLN will also depend, for example, on the type of assay (e.g., ELISA, enzyme activity, immunohistochemistry, imaging), the sample to be tested (e.g., serum, tumor tissue, urine), and other considerations known to those of skill in the art. The ULN or LLN can be used to define cut-offs between normal and abnormal. For example, a low level of a marker (e.g., LDH) can be defined as a marker level less than or equal to the ULN for that marker, with a high level being all values greater than the ULN. Cut-offs can also be defined as fractional amounts of the ULN. For example, a low level of a marker can be understood to be a level of about 0.5 ULN or less, 0.6 ULN or less, 0.7 ULN or less, 0.8 ULN or less, 0.9 ULN or less, 1.0 ULN or less, 1.1 ULN or less, 1.2 ULN or less, 1.3 ULN or less, 1.4 ULN or less, 1.5 ULN or less, 1.6 ULN or less, 1.7 ULN or less, 1.8 ULN or less, 1.9 ULN or less, 2.0 ULN or less, 2.5 ULN or less, 3.0 ULN or less, or 4.0 ULN or less. With the corresponding high level of the marker being any value greater than the low level. In certain embodiments, the presence of a low level of a marker in a subject sample as defined above can be indicative that a subject will or will not respond to a particular therapeutic intervention. In certain embodiments, the presence of a high level of a marker in a subject sample as defined above can be indicative that a subject will or will not respond to a particular therapeutic intervention.

Marker levels can also be further stratified, for example, into low, intermediate, and high based on the ULN value. For example, the presence of a low level of a marker in a subject sample as defined above can be indicative that a subject will or will not respond to a particular therapeutic intervention. An intermediate level of a marker, e.g., a range bracketed by any range within the values of 0.5 ULN, 0.6 ULN, 0.7 ULN, 0.8 ULN, 0.9 ULN, 1.0 ULN, 1.1 ULN, 1.2 ULN, 1.3 ULN, 1.4 ULN, 1.5 ULN, 1.6 ULN, 1.7 ULN, 1.8 ULN, 1.9 ULN, and 2.0 ULN, can be considered an intermediate range wherein the level of the marker may be indeterminate that a subject will or will not respond to a particular therapeutic intervention. A high level, greater than the intermediate level, would be indicative that a subject will or will not respond to a particular therapeutic intervention.

Similarly, cut-offs of ratios of LDH subunits or isoforms comparing the ULN, the LLN, or the median values to differentiate between high and low levels of hypoxia can be defined as any value or range bracketed by the values 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, or higher.

The “normal” level of expression of a marker is the level of expression of the marker in cells of a subject or patient not afflicted with cancer. In one embodiment, a “normal” level of expression refers to the level of expression of the marker under normoxic conditions in a subject not suffering from or known to be suffering from any diseases or conditions that include an ischemic or vascular regeneration component.

An “over-expression” or “high level of expression” of a marker refers to an expression level in a test sample that is greater than the standard error of the assay employed to assess expression, and is preferably at least 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3, 4, 5, 6, 7, 8, 9, or 10 times the expression level of the marker in a control sample (e.g., sample from a healthy subject not having the marker associated disease, i.e., cancer). In one embodiment, expression of a marker is compared to an average expression level of the marker in several control samples.

A “low level of expression” or “under-expression” of a marker refers to an expression level in a test sample that is less than 0.9, 0.8, 0.7, 0.6, 0.5, 0.4, 0.3, 0.2, or 0.1 times the expression level of the marker in a control sample (e.g., sample from a healthy subjects not having the marker associated disease, i.e., cancer). In one embodiment, expression of a marker is compared to an average expression level of the marker in several control samples.

As used herein, the term “identical” or “identity” is used herein in relation to amino acid and nucleic acid sequences and refers to any gene or protein sequence that bears at least 30% identity, more preferably 40%, 50%, 60%, 70%, 75%, 80%, 81%, 82%, 83%, 84%, 85%, 86%, 87%, 88%, 89%, 90%, 91%, 92%, 93%, 94%, and most preferably 95%, 96%, 97%, 98%, 99% or more identity to a known gene or protein sequence over the length of the comparison sequence. Protein or nucleic acid sequences with high levels of identity throughout the sequence can be said to be homologous. A “homologous” protein can also have at least one biological activity of the comparison protein. In general, for proteins, the length of comparison sequences will be at least 10 amino acids, preferably 10, 20, 30, 40, 50, 60, 70, 80, 90, 100, 150, 175, 200, 250, or at least 300 amino acids or more. For nucleic acids, the length of comparison sequences will generally be at least 25, 50, 100, 125, 150, 200, 250, 300, 350, 400, 450, 500, 550, 600, 650, 700, 800, or at least 850 nucleotides or more.

By “hybridize” is meant pair to form a double-stranded molecule between complementary polynucleotide sequences, or portions thereof, under various conditions of stringency. (See, e.g., Wahl and Berger Methods Enzymol. 152:399, 1987; Kimmel, Methods Enzymol. 152:507, 1987.) For example, stringent salt concentration will ordinarily be less than about 750 mM NaCl and 75 mM trisodium citrate, preferably less than about 500 mM NaCl and 50 mM trisodium citrate, and most preferably less than about 250 mM NaCl and 25 mM trisodium citrate. Low stringency hybridization can be obtained in the absence of organic solvent, e.g., formamide, while high stringency hybridization can be obtained in the presence of at least about 35% formamide, and most preferably at least about 50% formamide. Stringent temperature conditions will ordinarily include temperatures of at least about 30° C., more preferably of at least about 37° C., and most preferably of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In a preferred embodiment, hybridization will occur at 30° C. in 750 mM NaCl, 75 mM trisodium citrate, and 1% SDS. In a more preferred embodiment, hybridization will occur at 37° C. in 500 mM NaCl, 50 mM trisodium citrate, 1% SDS, 35% formamide, and 100 μg/ml denatured salmon sperm DNA (ssDNA). In a most preferred embodiment, hybridization will occur at 42° C. in 250 mM NaCl, 25 mM trisodium citrate, 1% SDS, 50% formamide, and 200 μg/ml ssDNA. Useful variations on these conditions will be readily apparent to those skilled in the art.

As used herein, the term “oxygen-sensitive pathway” is a cellular signaling pathway which is activated by hypoxia. Oxygen-sensitive pathways may be up-regulated by hypoxia. Alternatively, an oxygen-sensitive pathway may be down-regulated by hypoxia. Oxygen-sensitive pathways include, but are not limited to, HIF pathways (such as HIF1α pathways), VEGF pathways, and mTOR pathways. As used herein, the term “hypoxia-modulated gene” or “hypoxia-modulated polypeptide” refers to a gene or protein which is up-regulated or down-regulated by hypoxia.

As used herein, the term “HIF pathway” and “HIF pathway members” as used herein, describe proteins and other signaling molecules that are regulated by HIF-1 and HIF-2. Hypoxia-Inducible Factor 1 (HIF-1) is a transcription factor that has been shown to play an essential role in cellular responses to hypoxia. Upon hypoxic stimulation, HIF-1 has been shown to activate genes that contain Hypoxic Response Elements (HREs) in their promoters, and thus up-regulate a series of gene products that promote cell survival under conditions of low oxygen availability. The list of known HIF-responsive genes includes glycolytic enzymes (such as lactate dehydrogenase (LDH), enolase-1 (ENO-I), and aldolase A, glucose transporters (GLUT 1 and GLUT 3), vascular endothelial growth factor (VEGF), inducible nitric oxide synthase (NOS-2), and erythropoietin (EPO). The switch of the cell to anaerobic glycolysis, and the up-regulation of angiogenesis by VEGF is geared at maximizing cell survival under conditions of low oxygen tension by reducing the requirement for oxygen, and increasing vasculature to maximize oxygen delivery to tissues. The HIF-1 transcription complex has recently been shown to comprise a heterodimer of two basic helix-loop-helix proteins, HIF-1α and HIF-1β (also known as ARNT, Aryl Hydrocarbon Receptor Nuclear Translocator).

HIF-1α is a member of the basic-helix-loop-helix PAS domain protein family and is an approximately 120 kDa protein containing two transactivation domains (TAD) in its carboxy-terminal half and DNA binding activity located in the N-terminal half of the molecule. HIF-1α is constitutively degraded by the ubiquitin-proteosome pathway under conditions of normoxia, a process that is facilitated by binding of the von Hippel-Lindau (VHL) tumor suppressor protein to HIF-1α. Under conditions of hypoxia, degradation of HIF-1α is blocked and active HIF-1α accumulates. The subsequent dimerization of HIF-1α a with ARNT leads to the formation of active HIF transcription complexes in the nucleus, which can bind to and activate HREs on HIF-responsive genes.

As used herein, the term “VEGF pathway” and “VEGF pathway members” as used herein, describe proteins and other signaling molecules that are regulated by VEGF. For example, VEGF pathway members include VEGFR 1, 2, and 3; PECAM-1, LacCer synthase, and PLA2.

As used herein, the term “mTOR pathway” and “mTOR pathway members” as used herein, describe proteins and other signaling molecules that are regulated by mTOR. For example, mTOR pathway members include SK6, PDCD4, eIF4B, RPS6, eIF4, 4E-BP1, and eIF4E.

“Chemotherapeutic agent” is understood as a drug used for the treatment of cancer. Chemotherapeutic agents include, but are not limited to, small molecules and biologics (e.g., antibodies, peptide drugs, nucleic acid drugs).

As used herein, “detecting”, “detection” and the like are understood that an assay performed for identification of a specific analyte in a sample, e.g., a hypoxia modulated polypeptide or a hypoxia modulated gene in a sample. The amount of analyte or activity detected in the sample can be none or below the level of detection of the assay or method.

The terms “modulate” or “modulation” refer to upregulation (i.e., activation or stimulation), downregulation (i.e., inhibition or suppression) of a level, or the two in combination or apart. A “modulator” is a compound or molecule that modulates, and may be, e.g., an agonist, antagonist, activator, stimulator, suppressor, or inhibitor.

The term “expression” is used herein to mean the process by which a polypeptide is produced from DNA. The process involves the transcription of the gene into mRNA and the translation of this mRNA into a polypeptide. Depending on the context in which used, “expression” may refer to the production of RNA, protein, or both.

The terms “level of expression of a gene” or “gene expression level” refer to the level of mRNA, as well as pre-mRNA nascent transcript(s), transcript processing intermediates, mature mRNA(s) and degradation products, or the level of protein, encoded by the gene in the cell.

As used herein, “level of activity” is understood as the amount of protein activity, typically enzymatic activity, as determined by a quantitative, semi-quantitative, or qualitative assay. Activity is typically determined by monitoring the amount of product produced in an assay using a substrate that produces a readily detectable product, e.g., colored product, fluorescent product, radioactive product. For example, the isoforms of LDH in a sample can be resolved using gel electrophoresis. Lactate, nicotinamide adenine dinucleotide (NAD+), nitroblue tetrazolium (NBT), and phenazine methosulphate (PMS) can be added to assess LDH activity. LDH converts lactate to pyruvate and reduces NAD+ to NADH. The hydrogens from NADH are transferred by PMS to NBT reducing it to a purple formazan dye. The percentage of each LDH isoenzyme activity as well as the relative amount of each isoform to the other isoforms or total LDH can be determined, for example, by densitometry.

As used herein, “changed as compared to a control” sample or subject is understood as having a level of the analyte or diagnostic or therapeutic indicator (e.g., marker) to be detected at a level that is statistically different than a sample from a normal, untreated, or control sample Control samples include, for example, cells in culture, one or more laboratory test animals, or one or more human subjects. Methods to select and test control samples are within the ability of those in the art. An analyte can be a naturally occurring substance that is characteristically expressed or produced by the cell or organism (e.g., an antibody, a protein) or a substance produced by a reporter construct (e.g., β-galactosidase or luciferase). Depending on the method used for detection the amount and measurement of the change can vary. Changed as compared to a control reference sample can also include a change in one or more signs or symptoms associated with or diagnostic of disease, e.g., cancer. Determination of statistical significance is within the ability of those skilled in the art, e.g., the number of standard deviations from the mean that constitute a positive result.

As used herein, “binding” is understood as having at least a 10² or more, 10³ or more, preferably 10⁴ or more, preferably 10⁵ or more, preferably 10⁶ or more preference for binding to a specific binding partner as compared to a non-specific binding partner (e.g., binding an antigen to a sample known to contain the cognate antibody).

“Determining” as used herein is understood as performing an assay or using a diagnostic method to ascertain the state of someone or something, e.g., the presence, absence, level, or degree of a certain condition, biomarker, disease state, or physiological condition.

“Prescribing” as used herein is understood as indicating a specific agent or agents for administration to a subject.

As used herein, the terms “respond” or “response” are understood as having a positive response to treatment with a therapeutic agent, wherein a positive response is understood as having a decrease in at least one sign or symptom of a disease or condition (e.g., tumor shrinkage, decrease in tumor burden, inhibition or decrease of metastasis, improving quality of life (“QOL”), delay of time to (tumor) progression (“TTP”), increase of overall survival (“OS”), etc.), or slowing or stopping of disease progression (e.g., halting tumor growth or metastasis, or slowing the rate of tumor growth or metastasis). A response can also include an improvement in quality of life, or an increase in survival time or progression free survival.

Reference will now be made in detail to preferred embodiments of the invention. While the invention will be described in conjunction with the preferred embodiments, it will be understood that it is not intended to limit the invention to those preferred embodiments. To the contrary, it is intended to cover alternatives, modifications, and equivalents as may be included within the spirit and scope of the invention as defined by the appended claims.

Elesclomol (STA-4783) is disclosed in U.S. Pat. No. 7,795,313 (incorporated herein by reference) a compound represented by the formula N-malonyl-bis(N′-methyl-N′-thiobenzoylhydrazide) shown below

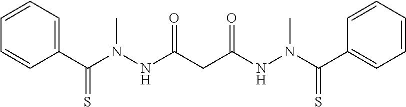 [SMILES: CN(NC(=O)CC(=O)NN(C)C(=S)c1ccccc1)C(=S)c1ccccc1]

Elesclomol has been tested or has been approved for testing in clinical trials for treatment of metastatic melanoma, prostate cancer, solid tumors, recurrent or persistent ovarian epithelial cancer, fallopian tube cancer, and primary peritoneal cancer. The half-life of elesclomol in humans has been determined to be about an hour when administered alone or with other chemotherapeutic agents (e.g., with paxitaxel or carboplatinin).

Elesclomol is more effective in treating disease, e.g., cancer, when administered to a patient with a cancer or tumor exhibiting low levels of hypoxia. In another embodiment, elesclomol is more effective in treating disease, e.g., cancer, when administered to a patient with a cancer or tumor exhibiting low levels of LDH.

I. Dosages and Modes of Administration

Techniques and dosages for administration vary depending on the type of compound (e.g., chemical compound, antibody, antisense, or nucleic acid vector) and are well known to those skilled in the art or are readily determined.

Therapeutic compounds of the present invention may be administered with a pharmaceutically acceptable diluent, carrier, or excipient, in unit dosage form. Administration may be parenteral, intravenous, subcutaneous, oral, or local by direct injection into the amniotic fluid. Administering an agent can be performed by a number of people working in concert. Administering an agent includes, for example, prescribing an agent to be administered to a subject and/or providing instructions, directly or through another, to take a specific agent, either by self-delivery, e.g., as by oral delivery, subcutaneous delivery, intravenous delivery through a central line, etc; or for delivery by a trained professional, e.g., intravenous delivery, intramuscular delivery, intratumoral delivery, etc.

The composition can be in the form of a pill, tablet, capsule, liquid, or sustained release tablet for oral administration; or a liquid for intravenous, subcutaneous, or parenteral administration; or a polymer or other sustained release vehicle for local administration.

Methods well known in the art for making formulations are found, for example, in “Remington: The Science and Practice of Pharmacy” (20th ed., ed. A. R. Gennaro, 2000, Lippincott Williams & Wilkins, Philadelphia, Pa.). Formulations for parenteral administration may, for example, contain excipients, sterile water, saline, polyalkylene glycols such as polyethylene glycol, oils of vegetable origin, or hydrogenated napthalenes. Biocompatible, biodegradable lactide polymer, lactide/glycolide copolymer, or polyoxyethylene-polyoxypropylene copolymers may be used to control the release of the compounds. Nanoparticulate formulations (e.g., biodegradable nanoparticles, solid lipid nanoparticles, liposomes) may be used to control the biodistribution of the compounds. Other potentially useful parenteral delivery systems include ethylene-vinyl acetate copolymer particles, osmotic pumps, implantable infusion systems, and liposomes. The concentration of the compound in the formulation varies depending upon a number of factors, including the dosage of the drug to be administered, and the route of administration.

The compound may be optionally administered as a pharmaceutically acceptable salt, disalt, or transition metal chelate or complex. See, e.g., U.S. Pat. No. 7,385,084 and PCT application No. PCT/US2009/61491, respectively.

Formulations for oral use include tablets containing the active ingredient(s) in a mixture with non-toxic pharmaceutically acceptable excipients. These excipients may be, for example, inert diluents or fillers (e.g., sucrose and sorbitol), lubricating agents, glidants, and anti-adhesives (e.g., magnesium stearate, zinc stearate, stearic acid, silicas, hydrogenated vegetable oils, or talc).

Formulations for oral use may also be provided as chewable tablets, or as hard gelatin capsules wherein the active ingredient is mixed with an inert solid diluent, or as soft gelatin capsules wherein the active ingredient is mixed with water or an oil medium.

The dosage and the timing of administering the compound depends on various clinical factors including the overall health of the subject and the severity of the symptoms of disease, e.g., cancer. In general, once a tumor is detected, administration of elesclomol is used to treat or prevent further progression of the tumor. Treatment can be continued for a period of time ranging from 1 to 100 days, more preferably 1 to 60 days, and most preferably 1 to 20 days, or until the remission of the tumor. It is understood that many chemotherapeutic agents are not administered daily, particularly agents with a long half-life. Therefore, an agent can be continually present without being administered daily. Dosages vary depending on each compound and the severity of the condition. Dosages can be titrated to achieve a steady-state blood serum concentration. Dosages can be interrupted or decreased in the presence of dose limiting toxicities.

### II. METHODS OF THE INVENTION

The instant invention provides methods of identifying a subject who will likely respond favorably to treatment with elesclomol by determining the level of hypoxia in a tumor, either by looking directly at markers within the tumor tissue or looking at markers in a peripheral sample from the subject, e.g., a bodily fluid such as blood, serum, plasma, lymph, urine, cerebrospinal fluid, or fecal matter, for the presence of one or more indicators of the level of hypoxia in the tumor.

The specific subject sample analyzed will depend, for example, on the site of the tumor. It is known that hypoxia drives angiogenesis in tumors, resulting in leaky blood vessels resulting in the presence of markers in circulation. Further, tumor growth and hypoxia are typically associated with necrosis and cell breakdown, resulting in cellular material in other bodily fluids or wastes. These readily accessible subject samples allow for the monitoring of the subject for the presence, or absence, of markers for hypoxia prior to and during the course of treatment.

Biopsies are routinely obtained for the purpose of cancer diagnosis, and solid tumors are frequently further resected prior to initiation of chemotherapy which also can be used for analysis to determine the level of hypoxia. Biopsy samples and resected tumor samples typically include at least some normal tissue adjacent to the tumor that can be used as a control.

In an embodiment of the invention, the level of hypoxia is a low level of hypoxia. In an embodiment of the invention, the level of LDH is a low level of LDH.

In one embodiment, the level of hypoxia is determined by detecting the level of one or more hypoxia-modulated polypeptides or using one or more methods such as imaging methods. In one embodiment, a hypoxia-modulated polypeptide is at least one isoform or subunit of lactate dehydrogenase (LDH), at least one isoform or subunit of hypoxia inducible factor (HIF), at least one pro-angiogenic form of vascular endothelial growth factor (VEGF), phosphorylated VEGF receptor (pKDR) 1, 2, or 3, neurolipin 1 (NRP-1), pyruvate dehydrokinase (PDH-K), and ornithine decarboxylase (ODC). In one embodiment, the isoform or subunit of LDH is LDHH, LDH5, LDH4, LDH3, LDH2, LDH1 or LDHM, or any combination thereof. In another embodiment, the isoform or subunit of LDH is LDH5. In another embodiment, the isoform of HIF is HIF-1α, HIF-1β, HIF-2α, and HIF-2β. In another embodiment, the pro-angiogenic isoform of VEGF is any one or combination of VEGF-A splice variants. Antibodies against prodrugs that localize in hypoxic regions (e.g., EF5, pimonidazole, etc.) can also be used to detect hypoxia. Tumor size can also be correlated with a level of hypoxia. A level of hypoxia can also be determined by PET scan. Functional imaging measuring blood flow in the tumor can be used as an indicator of hypoxia in the tissue. Direct measurement of hypoxia can be preformed by inserting a sensor into the tumor.

Methods to detect the protein and activity levels of markers of hypoxia, or hypoxia modulated polypeptides, are well known in the art. Antibodies against and kits for detection of hypoxia modulated polypeptides can be purchased from a number of commercial sources. Alternatively, using routine methods known in the art (e.g., immunization of animals, phage display, etc.) antibodies against one or more hypoxia modulated polypeptides or subunits or isoforms thereof can be made and characterized. Antibodies can be used for the detection of levels of hypoxia using ELISA, RIA, or other immunoassay methods, preferably automated methods, for the quantitative detection of proteins in samples of bodily fluids or homogenized solid samples. Alternatively, immunohistochemical methods can be used on tumor samples and tissue sections. Qualitative scoring methods and scanning methods to detect staining are known in the art. When qualitative scoring methods are used, it is preferred that two independent, blinded technicians, pathologists, or other skilled individuals analyze each sample with specific methods for resolving any significant disagreement in scoring, e.g., a third individual reviews the tissue sample. Many markers of hypoxia, including LDH, are enzymes. Enzymatic activity can be assayed in total, or for individual isoforms, for example, using in gel assays.

Alternatively, nucleic acid based methods of detection of levels of hypoxia are also well known in the art. Methods of designing primers and probes for quantitative reverse transcription real time (rt)PCR are known in the art. Methods for performing northern blots to detect RNA levels are known in the art. Nucleic acid detection methods can also include fluorescence in situ hybridization (FISH) and in situ PCR. Qualitative scoring methods and scanning methods to detect staining are known in the art. In another aspect, the present invention provides methods for the preselection of a subject for therapeutic treatment with elesclomol, wherein the subject has previously been found to have a low level of hypoxia. The invention also provides methods for the preselection of a subject for therapeutic treatment with an elesclomol by evaluating the results of an assessment of a sample from the subject for a low level of hypoxia.

Such determinations can be made based on a chart review of the level of hypoxia of the tumor of the subject. Inclusion criteria can include information being available regarding the cancer type, the specific treatment regimen with elesclomol, and the outcome to death or for a meaningful follow-up period which varies depending on the cancer type, e.g., metastatic or refractile cancers with poor prognoses requiring follow-up of weeks to months whereas cancers with less poor prognoses preferably having months to years of follow-up with subjects. In addition to information related to survival, information related to quality of life, side effects, and other relevant information can be considered when available. Exclusion criteria can include the presence of other diseases or conditions that could result in alteration of levels of hypoxia modulated peptides, e.g., ischemic heart or vascular disease, poor circulation, diabetes, macular degeneration, recent stroke, or other ischemic events or conditions. Other exclusion criteria can be selected based on the available samples and patient population, e.g., prior treatment with specific agents.

The subjects can be sorted into groups based on various criteria. Subjects who were treated with elesclomol for whom no levels of hypoxic markers were determined can be used as an unstratified control group to understand the efficacy of elesclomol on a treatment population not selected based on the level of hypoxia in the subject. Alternatively, the population analyzed in the study can be compared to historical control samples in which an unstratified population was analyzed for response to elesclomol.

Subjects for whom hypoxic levels were obtained can be divided into two or more groups having high and low level of hypoxia, optionally with a group of subjects with moderate levels of hypoxia, depending on the distribution of subjects. It is understood that subjects and samples can also be divided into other groups, e.g., survival time, treatment regimen with elesclomol, cancer type, previous failed treatments, etc. for analysis. Preferably, the same marker(s) of hypoxia is measured in each of the subjects, e.g., at least one isoform or subunit of lactate dehydrogenase (LDH) or hypoxia inducible factor (HIF); at least one pro-angiogenic form of vascular endothelial growth factor (VEGF), phosphorylated VEGF receptor (pKDR) 1, 2, or 3, GLUT-1, GLUT-2, neurolipin 1 (NRP-1), pyruvate dehydrokinase (PDH-K), and ornithine decarboxylase (ODC). Tumor size can also be a marker correlated with a level of hypoxia. A marker of a level of hypoxia can also be determined by PET scan. Further, it is preferred that the same type of subject sample, e.g., blood, serum, lymph, tumor tissue, etc., is tested for the presence of the marker for the level of hypoxia. It is understood that the level of hypoxia can be measured directly in the tumor sample, using quantitative, semi-quantitative, or qualitative immunohistochemical methods, immunological assays (e.g., ELISA assay); reverse transcription PCR assays, particularly quantitative PCR methods, e.g., real time PCR; northern blot assays, enzyme activity assays (e.g., for lactate dehydrogenase activity, for kinase activity); and in situ hybridization assay (e.g., fluorescence in situ hybridization (FISH) assay). Antibodies against prodrugs that localize in hypoxic regions (e.g., EF5, pimonidazole, etc.) can also be used as markers to detect hypoxia. Functional imaging measuring bloodflow in the tumor can be used as a marker of hypoxia in the tissue. Direct measurement of hypoxia can be preformed to provide a marker for hypoxia by inserting a sensor into the tumor. Again, it is preferred that the same method of determining the level of the marker of hypoxia is used for all samples, particularly when qualitative assessment methods are used.

Outcomes of subjects based on the level of hypoxia can be analyzed to determine if the outcome between the two groups is different. Outcomes can further be compared to a non-stratified group treated with elesclomol. Methods for statistical analysis and determination of statistical significance are within the ability of those of skill in the art. Methods to determine appropriate cut-off levels for selection of treatment criteria are within the ability of those of skill in the art. The analysis demonstrates that subjects with a low level of hypoxia have a better response, e.g., one or more of longer time to failure, longer survival time, better quality of life, decreased tumor size, better quality of life, better tolerance of elesclomol, etc., as compared to subjects with a high level of hypoxia.

In another aspect, the present invention provides methods for the preselection of a subject for therapeutic treatment with elesclomol, wherein the subject has previously been found to have a low level of hypoxia. The invention also provides methods for the preselection of a subject for therapeutic treatment with elesclomol by evaluating the results of an assessment of a sample from the subject for a modulated level of hypoxia wherein the subject is found to have a low level of hypoxia. Such determinations can be made based on the level of hypoxia observed in historical samples. An analysis using samples collected from subjects during treatment can be performed to determine the efficacy of elesclomol for the treatment of cancer based on the level of hypoxia of the tumor based on markers assessed during the treatment of the subjects. Inclusion criteria are information being available regarding the cancer type, the specific treatment regimen with elesclomol, and the outcome to death or for a meaningful follow-up period which varies depending on the cancer type, e.g., metastatic or refractile cancers with poor prognoses requiring follow-up of weeks to months whereas cancers with less poor prognoses preferably having months to years of follow-up with subjects. In addition to information related to survival, information related to quality of life, side effects, and other relevant information is considered when available. Exclusion criteria can include the presence of other diseases or conditions that could result in alteration of levels of hypoxia modulated peptides, e.g., ischemic heart or vascular disease, poor circulation, diabetes, macular degeneration, recent stroke, or other ischemic events or conditions. Other exclusion criteria can be selected based on the available samples and patient population, e.g., prior treatment with specific agents.

The samples can be analyzed for the level of hypoxia. Preferably, all of the samples are the same type or types, e.g., blood, plasma, lymph, tumor tissue. Depending on the availability of subject samples, the analysis can be performed using two (or more) subject sample types, e.g., serum and tumor tissue. Various portions of the tumor tissue can also be analyzed when sufficient material is available, e.g., adjacent to the necrotic core, in the center of the tumor, adjacent to or including tumor vasculature, adjacent to normal tissue, etc. One or more markers of hypoxia can be measured in each of the subjects, e.g., at least one isoform or subunit of lactate dehydrogenase (LDH) or hypoxia inducible factor (HIF); at least one pro-angiogenic form of vascular endothelial growth factor (VEGF), phosphorylated VEGF receptor (pKDR) 1, 2, or 3, GLUT-1, GLUT-2, neurolipin 1 (NRP-1), pyruvate dehydrokinase (PDH-K), and ornithine decarboxylase (ODC). Enzymatic assays of markers can be performed. Tumor size can also be a marker correlated with a level of hypoxia. A marker of a level of hypoxia can also be determined by PET scan. Antibodies against prodrugs that localize in hypoxic regions (e.g., EF5, pimonidazole, etc.) can also be used as markers to detect hypoxia. Functional imaging measuring bloodflow in the tumor can be used as a marker of hypoxia in the tissue. Direct measurement of hypoxia can be preformed to provide a marker for hypoxia by inserting a sensor into the tumor.

Further, it is preferred that the same type of subject sample, e.g., blood, serum, lymph, tumor tissue, etc., is tested for the presence of the marker for the level of hypoxia. It is understood that the level of hypoxia could have been measured directly in the tumor sample, using quantitative, semi-quantitative, or qualitative immunohistochemical methods, immunological assays (e.g., ELISA assay); reverse transcription PCR assays, particularly quantitative PCR methods, e.g., real time PCR; northern blot assays, enzyme activity assays (e.g., for lactate dehydrogenase activity, for kinase activity); and in situ hybridization assay (e.g., fluorescence in situ hybridization (FISH) assay). Again, it is preferred that the same method of determining the level of the marker of hypoxia is used for all samples, particularly when qualitative assessment methods are used.

In another aspect, the present invention provides methods for treating a cancer with elesclomol in a subject having a low level of hypoxia. The methods include administering elesclomol to the subject having a cancer or susceptible to a cancer who further has a low level of hypoxia, thereby treating the cancer. Other methods include administering elesclomol and at least one chemotherapeutic agent to the subject having a cancer or susceptible to a cancer wherein the subject has a low level of hypoxia, thereby treating the cancer. In certain embodiments, the subject has previously been treated with a chemotherapeutic agent.

Other methods include methods of treating a subject who has cancer by prescribing to the subject an effective amount of elesclomol, wherein the subject has previously been found to have a low level of hypoxia. As used herein, the term “prescribing” is understood as indicating a specific agent or agents for administration to a subject. Furthermore, the present invention also includes methods of increasing the likelihood of effectively treating a subject having cancer by administering a therapeutically effective amount of a composition comprising elesclomol to the subject, wherein the subject has previously been found to have a low level of hypoxia.

It is understood that diagnosis and treatment of a complex disease such as cancer is not performed by a single individual, test, agent, or intervention. For example, a subject may meet with a primary care physician to express a concern and be referred to an oncologist who will request tests that are designed, carried out, and analyzed by any of a number of individuals, but not limited to, radiologists, radiology technicians, physicists, phlebotomists, pathologists, laboratory technicians, and radiation, clinical, and surgical oncologists. Selection, dosing, and administration of agents to a subject diagnosed with cancer will be performed by any of a number of individuals including, but not limited to, radiologists, radiology technicians, physicists, pathologists, infusion nurses, pharmacists, and radiation, clinical, and surgical oncologists. Therefore, it is understood that within the terms of the invention, identifying a subject as having a specific level of hypoxia can include any of a number of acts including, but not limited to, performing a test and observing a result that is indicative of a subject having a specific level of hypoxia; reviewing a test result of a subject and identifying the subject as having a specific level of hypoxia; reviewing documentation on a subject stating that the subject has a specific level of hypoxia and identifying the subject as the one discussed in the documentation by confirming the identity of the subject, e.g., by an identification card, hospital bracelet, asking the subject for his/her name and/or other personal information to confirm the subjects identity.

Similarly, administering an agent can be performed by a number of people working in concert. Administering an agent includes, for example, prescribing an agent to be administered to a subject and/or providing instructions, directly or through another, to take a specific agent, either by self-delivery, e.g., as by oral delivery, subcutaneous delivery, intravenous delivery through a central line, etc; or for delivery by a trained professional, e.g., intravenous delivery, intramuscular delivery, intratumoral delivery, etc.

In another aspect, the present invention provides methods for treating a cancer. The methods include administering elesclomol to the subject having a cancer or susceptible to a cancer wherein the subject has a low level of hypoxia, thereby treating the cancer. Other methods include administering elesclomol and at least one chemotherapeutic agent to the subject having a cancer or susceptible to a cancer, thereby treating the cancer.

Cancers that may be treated or prevented using the methods of the invention include, for example, acoustic neuroma, acute leukemia, acute lymphocytic leukemia, acute myelocytic leukemia (monocytic, myeloblastic, adenocarcinoma, angiosarcoma, astrocytoma, myelomonocytic and promyelocytic), acute T-cell leukemia, basal cell carcinoma, bile duct carcinoma, bladder cancer, brain cancer, breast cancer, bronchogenic carcinoma, cervical cancer, chondrosarcoma, chordoma, choriocarcinoma, chronic leukemia, chronic lymphocytic leukemia, chronic myelocytic (granulocytic) leukemia, chronic myleogeneous leukemia, colon cancer, colorectal cancer, craniopharyngioma, cystadenocarcinoma, diffuse large B-cell lymphoma, dysproliferative changes (dysplasias and metaplasias), embryonal carcinoma, endometrial cancer, endotheliosarcoma, ependymoma, epithelial carcinoma, erythroleukemia, esophageal cancer, estrogen-receptor positive breast cancer, essential thrombocythemia, Ewing's tumor, fibrosarcoma, follicular lymphoma, germ cell testicular cancer, glioma, heavy chain disease, hemangioblastoma, hepatoma, hepatocellular cancer, hormone insensitive prostate cancer, leiomyosarcoma, liposarcoma, lung cancer, lymphagioendotheliosarcoma, lymphangiosarcoma, lymphoblastic leukemia, lymphoma (Hodgkin's and non-Hodgkin's), malignancies and hyperproliferative disorders of the bladder, breast, colon, lung, ovaries, pancreas, prostate, skin and uterus, lymphoid malignancies of T-cell or B-cell origin, leukemia, lymphoma, medullary carcinoma, medulloblastoma, melanoma, meningioma, mesothelioma, multiple myeloma, myelogenous leukemia, myeloma, myxosarcoma, neuroblastoma, non-small cell lung cancer, oligodendroglioma, oral cancer, osteogenic sarcoma, ovarian cancer, pancreatic cancer, papillary adenocarcinomas, papillary carcinoma, pinealoma, polycythemia vera, prostate cancer, rectal cancer, renal cell carcinoma, retinoblastoma, rhabdomyosarcoma, sarcoma, sebaceous gland carcinoma, seminoma, skin cancer, small cell lung carcinoma, solid tumors (carcinomas and sarcomas), small cell lung cancer, stomach cancer, squamous cell carcinoma, synovioma, sweat gland carcinoma, thyroid cancer, Waldenstrom's macroglobulinemia, testicular tumors, uterine cancer and Wilms' tumor. Other cancers include primary cancer, metastatic cancer, oropharyngeal cancer, hypopharyngeal cancer, liver cancer, gallbladder cancer, small intestine cancer, urinary tract cancer, kidney cancer, urothelium cancer, female genital tract cancer, uterine cancer, gestational trophoblastic disease, male genital tract cancer, seminal vesicle cancer, testicular cancer, germ cell tumors, endocrine gland tumors, thyroid cancer, adrenal cancer, and pituitary gland cancer, hemangiomas, sarcomas arising from bone and soft tissues; Kaposi's sarcoma, nerve cancer, ocular cancer, and meningial cancer, glioblastomas, neuromas, Schwannomas, solid tumors arising from hematopoietic malignancies such as leukemias, metastatic melanoma, recurrent or persistent ovarian epithelial cancer, fallopian tube cancer, primary peritoneal cancer, gastrointestinal stromal tumors, colorectal cancer, gastric cancer, melanoma, glioblastoma multiforme, non-squamous non-small-cell lung cancer, malignant glioma, epithelial ovarian cancer, primary peritoneal serous cancer, metastatic liver cancer, neuroendocrine carcinoma, refractory malignancy, triple negative breast cancer, HER2 amplified breast cancer, squamous cell carcinoma of the head and neck (SCCHN), nasopharageal cancer, oral cancer, biliary tract, hepatocellular carcinoma, non-medullary thyroid carcinoma, recurrent glioblastoma multiforme, neurofibromatosis type 1, CNS cancer, liposarcoma; leiomyosarcoma; salivary gland cancer, mucosal melanoma; acral/lentiginous melanoma, paraganglioma, and pheochromocytoma.

### III. KITS OF THE INVENTION

The invention also provides for kits to practice the methods of the invention. For example, a kit can include elesclomol and an instruction for administration of elesclomol to a subject having cancer with a low level of hypoxia. In another embodiment, the subject has cancer with a low level of lactate dehydrogenase (LDH). In an embodiment, the instruction provides that elesclomol is a second line therapy. In another example, the kits of the invention may contain reagents for determining the level of LDH in a sample from a subject and may further contain instructions for treating a subject with cancer with a low level of hypoxia with elesclomol.

### EXAMPLES

### Example 1

### Selection of Subjects for Treatment with Elesclomol Based on Hypoxic Level

A subject is diagnosed with cancer based on a series of clinically accepted diagnostic criteria including imaging, immunohistochemistry, hematological analyses, and physical examination. The immunohistochemical analysis includes staining for the presence of one or more hypoxic markers in the biopsy sample. Further, or alternatively, a serum sample is tested for the presence of one or more hypoxic markers.

A subject is identified as having a low level of a hypoxic marker in serum and/or in the tumor. The subject is selected for treatment with elesclomol. The subject is treated with elesclomol and monitored for therapeutic response as well as the presence of side effects. Therapy is continued as long as it is sufficiently tolerated and a benefit to the subject is observed as determined by the subject, the treating physician, the caregiver, and/or other qualified individual.

### Example 2

### Selection of Subjects not to be Treated with Elesclomol Based on Hypoxic Level

A subject is diagnosed with cancer based on a series of clinically accepted diagnostic criteria including imaging, immunohistochemistry, hematological analyses, and physical examination. The immunohistochemical analysis includes staining for the presence of one or more hypoxic markers in the biopsy sample. Further, or alternatively, a serum sample is tested for the presence of one or more hypoxic markers.

A subject is identified as having a high level of a hypoxic marker in serum and/or in the tumor. A treatment regimen not including elesclomol is selected for the subject.

### Example 3

### Characterization of Treatment Outcomes Based on Chart Review

A chart review analysis is performed to determine the efficacy of elesclomol for the treatment of cancer based on the level of hypoxia of the tumor based on markers assessed during the treatment of the subjects. Inclusion criteria are information being available regarding the cancer type, the specific treatment regimen with elesclomol, and the outcome over a meaningful follow-up period which varies depending on the cancer type, e.g., metastatic or refractile cancers with poor prognoses requiring follow-up of weeks to months (e.g., until death, until tumor progression, until administration of new therapeutic intervention) whereas cancers with less poor prognoses preferably having months to years of follow-up with subjects (e.g., until tumor progression, until administration of new therapeutic intervention, to an arbitrary end point). In addition to information related to survival, information related to quality of life, side effects, and other relevant information is considered when available. Exclusion criteria can include the presence of other diseases or conditions that could result in alteration of levels of hypoxia modulated peptides, e.g., ischemic heart or vascular disease, poor circulation, diabetes, macular degeneration, recent stroke, recent surgery, or other ischemic events or conditions. Other exclusion criteria can be selected based on the available samples and patient population, e.g., prior treatment with specific agents.

The subjects can be sorted into groups based on various criteria. Subjects who were treated with elesclomol for whom no levels of hypoxic markers were determined can be used as an unstratified control group to understand the efficacy of elesclomol on a treatment population not selected based on the level of hypoxia in the subject/tumor. Alternatively, the population analyzed in the study for which hypoxia levels (e.g., LDH marker levels) can be compared to historical control samples in which an unstratified population was analyzed for response to elesclomol.

Subjects for whom hypoxic levels are available in chart records are divided into two or more groups having high and low levels of hypoxia, optionally with a group of subjects with moderate levels of hypoxia, depending on the distribution of subjects. It is understood that subjects and samples can also be divided into other groups, e.g., survival time, treatment regimen with elesclomol, cancer type, previous failed treatments, etc. for analysis. Preferably, the same marker(s) of hypoxia is measured in each of the subjects, e.g., at least one isoform or subunit of lactate dehydrogenase (LDH) or hypoxia inducible factor (HIF); at least one pro-angiogenic form of vascular endothelial growth factor (VEGF), phosphorylated VEGF receptor (pKDR) 1, 2, or 3; neurolipin 1 (NRP-1), pyruvate dehydrokinase (PDH-K), and ornithine decarboxylase (ODC). Antibodies against prodrugs that localize in hypoxic regions (e.g., EF5, pimonidazole, etc.) can also be markers hypoxia. Functional imaging measuring bloodflow in the tumor can be used as a marker of hypoxia in the tissue. Direct measurement of hypoxia can be a marker and can be preformed by inserting a sensor into the tumor. Tumor size can also be a marker correlated with hypoxia. Further, it is preferred that the same type of subject sample, e.g., blood, serum, lymph, tumor tissue, etc., is tested for the presence of the marker for the level of hypoxia. It is understood that the level of hypoxia can be measured directly in the tumor sample, using quantitative, semi-quantitative, or qualitative immunohistochemical methods, immunological assays (e.g., ELISA assay); reverse transcription PCR assays, particularly quantitative PCR methods, e.g., real time PCR; northern blot assays, enzyme activity assays (e.g., for lactate dehydrogenase activity, for kinase activity); and in situ hybridization assay (e.g., fluorescence in situ hybridization (FISH) assay). Again, it is preferred that the same method of determining the level of the marker of hypoxia is used in all samples, particularly when qualitative assessment methods are used.

Outcomes of subjects based on the level of hypoxia are analyzed to determine if the outcome between the two groups is different. Outcomes can further be compared to a non-stratified group treated with elesclomol. Statistical methods can be used to select appropriate cut-off levels to identify subjects most likely to benefit from treatment with elesclomol based on the level of one or more hypoxic markers. Methods for statistical analysis and determination of statistical significance are within the ability of those of skill in the art. The analysis demonstrates that subjects with a low level of hypoxia have a better response, e.g., one or more of longer time to failure, longer survival time, better quality of life, decreased tumor size, better tolerance of elesclomol, etc., as compared to subjects with a high level of hypoxia.

### Example 4

### Characterization of Treatment Outcomes Based on Historical Samples

An analysis using samples collected from subjects during treatment is performed to determine the efficacy of elesclomol for the treatment of cancer based on the level of hypoxia of the tumor based on markers assessed prior to and/or during the treatment of the subjects. Inclusion criteria are information being available regarding the cancer type, the specific treatment regimen with elesclomol, and the outcome for a meaningful follow-up period which varies depending on the cancer type, e.g., metastatic or refractile cancers with poor prognoses requiring follow-up of weeks to months (e.g., until death, until tumor progression, until administration of new therapeutic intervention) whereas cancers with less poor prognoses preferably having months to years of follow-up (e.g., until tumor progression, until administration of new therapeutic intervention, to an arbitrary end point) with subjects. In addition to information related to survival, information related to quality of life, side effects, and other relevant information is considered when available. Exclusion criteria include the presence of other diseases or conditions that could result in alteration of levels of hypoxia modulated peptides, e.g., ischemic heart or vascular disease, poor circulation, diabetes, macular degeneration, recent stroke, or other ischemic events or conditions. Other exclusion criteria can be selected based on the available samples and patient population, e.g., prior treatment with specific agents.

The samples are analyzed for the level of hypoxia based on the amount or activity of a marker present. Preferably, all of the samples are the same type or types, e.g., blood, plasma, lymph, urine, tumor tissue. Depending on the availability of subject samples, the analysis can be performed using two (or more) subject sample types, e.g., serum and tumor tissue. Various portions of the tumor tissue can also be analyzed when sufficient material is available, e.g., adjacent to the necrotic core, in the center of the tumor, adjacent to or including tumor vasculature, adjacent to normal tissue, etc. One or more markers of hypoxia are measured in each of the subjects, e.g., at least one isoform or subunit of lactate dehydrogenase (LDH) or hypoxia inducible factor (HIF); at least one pro-angiogenic form of vascular endothelial growth factor (VEGF), phosphorylated VEGF receptor (pKDR) 1, 2, or 3, neurolipin 1 (NRP-1), pyruvate dehydrokinase (PDH-K), and ornithine decarboxylase (ODC). Antibodies against prodrugs that localize in hypoxic regions (e.g., EF5, pimonidazole, etc.) can also be markers hypoxia. Functional imaging measuring bloodflow in the tumor can be used as a marker of hypoxia in the tissue. Direct measurement of hypoxia can be a marker and can be preformed by inserting a sensor into the tumor. Tumor size can also be a marker correlated with hypoxia. Further, it is preferred that the same type of subject sample, e.g., blood, serum, lymph, urine, tumor tissue, etc., is tested for the presence of the marker for the level of hypoxia. It is understood that the level of hypoxia can be measured directly in the tumor sample, using quantitative, semi-quantitative, or qualitative immunohistochemical methods, immunological assays (e.g., ELISA assay); reverse transcription PCR assays, particularly quantitative PCR methods, e.g., real time PCR; northern blot assays, enzyme activity assays (e.g., for lactate dehydrogenase activity, for kinase activity); and in situ hybridization assay (e.g., fluorescence in situ hybridization (FISH) assay). Again, it is preferred that the same method of determining the level of the marker of hypoxia is used in all samples, particularly when qualitative assessment methods are used.

Subjects are divided into two or more groups having high and low level of hypoxia, optionally with a group of subjects with moderate levels of hypoxia, depending on the distribution of subjects. It is understood that subjects and samples can also be divided into other groups, e.g., survival time, treatment regimen with elesclomol, cancer type, previous failed treatments, etc. for analysis.

Outcomes of subjects based on the level of hypoxia are analyzed to determine if the outcome between the two groups is different. Outcomes can further be compared to a non-stratified group treated with elesclomol, e.g., a historical group provided by another study. Statistical methods can be used to select appropriate cut-off levels to identify subjects most likely to benefit from treatment with elesclomol based on the level of one or more hypoxic markers. Methods for statistical analysis and determination of statistical significance are within the ability of those of skill in the art. The analysis demonstrates that subjects with a low level of hypoxia have a better response, e.g., one or more of longer time to failure, longer survival time, better quality of life, decreased tumor size, better tolerance of elesclomol, delayed time to progression, etc., as compared to subjects with a high level of hypoxia. The results indicate that subjects with low levels of hypoxia should be treated with elesclomol and those with high levels of hypoxia should not be treated with elesclomol.

### Example 5

### Trial to Demonstrate Improved Efficacy of Elesclomol in Subjects with a High Level of Hypoxia

Subjects diagnosed with solid tumors are recruited for a study to determine the efficacy of elesclomol in the treatment of solid tumors, preferably tumors from the same tissue origin, e.g., breast, prostate, lung, liver, brain, colorectal, etc. Inclusion criteria include the presence of a solid tumor. Exclusion criteria include the presence of an ischemia related disease or disorder including, e.g., ischemic heart or vascular disease, poor circulation, diabetes, macular degeneration, recent stroke, or other ischemic events or conditions; or surgery planned during the duration of the trial. Blood and tumor samples are collected for analysis of levels of hypoxia by determining the expression level or activity level of one or more markers of hypoxia, e.g., at least one isoform or subunit of lactate dehydrogenase (LDH) or hypoxia inducible factor (HIF); at least one pro-angiogenic form of vascular endothelial growth factor (VEGF), phosphorylated VEGF receptor (pKDR) 1, 2, or 3, neurolipin 1 (NRP-1), pyruvate dehydrokinase (PDH-K), and ornithine decarboxylase (ODC). Antibodies against prodrugs that localize in hypoxic regions (e.g., EF5, pimonidazole, etc.) can also be markers hypoxia. Functional imaging measuring bloodflow in the tumor can be used as a marker of hypoxia in the tissue. Direct measurement of hypoxia can be a marker and can be preformed by inserting a sensor into the tumor. Tumor size can also be a marker correlated with hypoxia. Depending on the tumor site, other subject samples can be collected, e.g., fecal matter in subjects with colorectal cancer, urine for subjects with kidney or bladder cancer, cerebrospinal fluid in subjects with brain cancer, etc. by assaying the same markers. Additional samples for analysis can be collected during the course of the study. Complete medical histories are also obtained when not otherwise available.

All subjects are treated with elesclomol, either alone or in combination with one or more additional chemotherapeutic agents. The number regimens used will depend on the size of the study, the number of subjects available, the time frame of the study, etc. The number of regimens is selected to allow the study to be sufficiently powered to provide meaningful results. Subjects are monitored for response to the agent throughout the trial, at the end of the trial, and at regular intervals after the conclusion of the trial using routine methods including, but not limited to, e.g., imaging, hematology, and physical examination. Treatment may be discontinued for non-responsive subjects or for with intolerable side effects. Preferably, the subjects continue to be monitored for outcomes beyond the formal end of the trial. Subjects with a positive response to the treatment regimen can be continued on the regimen at the discretion of the attending physician after the formal conclusion of the trial.

An analysis of the samples collected from subjects prior to and optionally during treatment is performed to determine the efficacy of elesclomol for the treatment of cancer based on the level of hypoxia of the tumor based on markers assessed prior to and optionally during the treatment of the subjects. The analysis can be performed at the conclusion of the trial, or the analysis can be performed prior to the conclusion of the trial with the results being blinded or not disclosed to the treating physicians. Preferably, the analysis for hypoxia level is determined during the course of the trial to insure that a sufficient number of subjects with high or low hypoxia levels were enrolled in the study to allow for sufficient power of the study to provide a conclusive outcome.

Outcomes of subjects based on the level of hypoxia are analyzed to determine if the outcome between the two groups is different. Outcomes can further be compared to a non-stratified group treated with elesclomol, e.g., a historical group provided by another study. Samples can be analyzed to confirm the correlation of the level of hypoxia in the tumor to the level of hypoxia in the peripherally collected sample (e.g., blood, urine, cerebrospinal fluid). Statistical methods can be used to select appropriate cut-off levels to identify subjects most likely to benefit from treatment with elesclomol based on the level of one or more hypoxic markers. Methods for statistical analysis and determination of statistical significance are within the ability of those of skill in the art. The analysis demonstrates that subjects with a low level of hypoxia have a better response, e.g., one or more of longer time to failure, longer survival time, better quality of life, decreased tumor size, better tolerance of elesclomol, etc., as compared to subjects with a high level of hypoxia. Therefore, subjects with lower levels of hypoxia should be selected for treatment with elesclomol, and those with higher levels should be administered other agents.

### Example 6

### Study to Demonstrate Improved Efficacy of Elesclomol in Subjects with Lung Cancer with a Low Level of LDH

Multiple clinical trials have been performed to demonstrate the efficacy of elesclomol in the treatment of lung cancer. For example, a Phase II study was performed to test elesclomol in combination with paclitaxel and carboplatin, as compared to paclitaxel and carboplatin alone, in 86 subjects with previously untreated non-small cell lung carcinoma (NSCLC) and non-resectable, locally advanced, or metastatic disease (Stage IIIB or IV) were randomized 1:1 into treatment groups for treatment with carboplatnin plus elesclomol (CP+E) or carboplatnin alone (CP). Subjects were dosed intravenously with 200 mg/m² of paclitaxel and AUC=6 of carboplatnin either with or without 266 mg/m² of elesclomol every three weeks for six cycles, likely resulting in complete clearance of elesclomol between doses. The results of this study are shown below.

Median Progression Free Survival (months)

| LDH level | CP + E | CP alone |
|---|---|---|
| Low | 4.6 (95% CI: 1.2-6.3) | 3.1 (95% CI: 2.2-4.6) |
| High | 2.8 (95% CI: 1.7-3.6) | 6.3 (95% CI: 4.8-8.0) |

Low LDH NSCLC patients receiving elesclomol had a median PFS of 4.6 months as compared to the control group of 3.1 months whereas, in the high LDH NSCLC patient's PFS relative magnitudes were flipped, with median PFS values of 2.8 and 6.3 months for the elesclomol receiving patients and control patient, respectively. These data demonstrate that a subject having a low level of LDH (equal to or less than the ULN) should be selected for treatment with elesclomol, and those with a high level of LDH should be selected against for treatment with elesclomol. These data further demonstrate that a subject having a low level of LDH (equal to or less than the ULN) is likely to benefit from treatment with elesclomol, and a subject having a high level of LDH (equal to or less than the ULN) is less likely or not likely to benefit from treatment with elesclomol.

### Example 7

### Study to Demonstrate Improved Efficacy of Elesclomol in Subjects with Melanoma with a Low Level of LDH

Multiple clinical trials have been performed to demonstrate the efficacy of elesclomol in the treatment of melanoma. For example, a Phase I/II study was performed to test elesclomol in combination with paclitaxel in patients with Stage IV Metastatic melanoma. Subjects were dosed intravenously with 80 mg/m² of paclitaxel either with or without 213 mg/m² of elesclomol once per week for the first three weeks, followed by one week off (i.e., a 28 day cycle with dosing on days 1, 8, and 14) with the 28 day dosing cycle repeated until tumor progression, likely resulting in complete clearance of elesclomol between doses. The study showed median PFS values were 7.1 and 3.5 months, respectively for treatment and control patients in the low LDH group, but 1.7 and 1.6 months, respectively for the high LDH patients (see table, below).

Median Progression Free Survival (months)

| LDH level | P + E | P alone |
|---|---|---|
| Low | 7.1 (95% CI: 4.4-9.0) | 3.5 (95% CI: 1.6-7.3) |
| High | 1.7 (95% CI: 1.6-3.7) | 1.6 (95% CI: 1.0-3.0) |

Further, a Phase III study was performed to test elesclomol in combination with paclitaxel versus paclitaxel alone in chemotherapy-naïve subjects with Stave IV Metastatic melanoma. Subjects with metastatic melanoma were randomized with a ratio of 1:1 to either the treatment arm (elesclomol 213 mg/m² in combination with paclitaxel 80 mg/m²) or the control arm (paclitaxel 80 mg/m² alone) once per week for the first three weeks, followed by one week off (i.e., a 28 day cycle with dosing on days 1, 8, and 14) with the 28 day dosing cycle repeated until tumor progression, likely resulting in complete clearance of elesclomol between doses. The population enrolled into the study was to have baseline LDH levels limited to 2 times upper limit of normal (ULN) values. Results of this trial are shown in the tables, below.

Median Progression Free Survival (months)

| LDH level | P + E | P alone |
|---|---|---|
| Low | 3.7 (95% CI: 3.4-5.4) | 2.1 (95% CI: 1.9-3.6) |
| High | 1.8 (95% CI: 1.7-2.1) | 1.9 (95% CI: 1.8-2.6) |

### Summary of Progression-Free Survival (PFS) Analysis

| | | High | Normal | Low |
|---|---|---|---|---|
| | ITT | LDH | LDH | LDH |
| | Population | (>1 × ULN) | (≦1 × ULN) | (≦0.8 × ULN) |
| Number of | | | | |
| Subjects | | | | |
| Elesclomol + | 325 | 112 | 213 | 142 |
| paclitaxel | | | | |
| Paclitaxel alone | 326 | 108 | 218 | 139 |
| Median | | | | |
| (months) PFS | | | | |
| Elesclomol + | 3.4 | 1.8 | 3.7 | 3.7 |
| paclitaxel | | | | |
| Paclitaxel alone | 1.9 | 1.9 | 2.1 | 2.1 |
| Hazard | 0.89 | 1.13 | 0.76 | 0.71 |
| Ratio^{[a]} | | | | |
| p-value^{[a]} | 0.2076 | 0.4229 | 0.0264 | 0.0245 |

^{[a]}Based on Cox regression model
ITT = intent-to-treat;
LDH = lactate dehydrogenase;
PFS = progression-free survival;
ULN = upper limit of normal

In the overall ITT population, the primary endpoint PFS was not statistically significantly different in treatment versus control arms (HR=0.89, p=0.2076), nor was there a difference in the high LDH population (HR=1.13, p=0.42). In contrast, in subjects with normal and low baseline LDH levels, PFS was statistically significantly improved (HR=0.76, p=0.026 and HR=0.71, p=0.0245, respectively) in treatment group as compared to the control group.

The table below presents a summary of the OS analyses for the ITT population and for High, Normal, and Low LDH groups. In the total population, median OS is similar for treatment and control arms (10.6 and 11.7 months, respectively), with an HR of 1.21 and p=0.0996. However, OS analysis per baseline LDH indicates that treatment with elesclomol in combination with paclitaxel has differential outcomes in different populations. In subjects with high baseline LDH, median OS was 6.0 and 8.1 months for treatment and control arms, respectively (HR=1.41, p=0.0406). In subjects with normal baseline LDH, median OS for treatment and control arms was 13.6 and 14.5 months (HR=1.04, p=0.7853). In subjects with low baseline LDH levels, median OS was not achieved for either the arm treated with elesclomol in combination with paclitaxel or the arm treated with paclitaxel alone (HR=0.96, p=0.8419).

### Summary of Overall Survival Analyses—ITT Population

| | ITT | High LDH | Normal LDH | Low LDH |
|---|---|---|---|---|
| | Population | (>1 × ULN) | (≦1 × ULN) | (≦0.8 × ULN) |
| Number of | | | | |
| Subjects | | | | |
| Elesclomol + | 325 | 112 | 213 | 142 |
| paclitaxel | | | | |
| Paclitaxel alone | 326 | 108 | 218 | 139 |
| % Overall | 52 | 30 | 63 | 66 |
| Survival Events | | | | |
| Censored | | | | |
| Median | | | | |
| (months) | | | | |
| Overall | | | | |
| Survival | | | | |
| Elesclomol + | 10.6 | 6.0 | 13.9 | Not Ach. |
| paclitaxel | | | | |
| Paclitaxel alone | 11.7 | 8.1 | 14.5 | Not Ach. |
| Hazard | 1.21 | 1.41 | 1.04 | 0.96 |
| Ratio^{[a]} | | | | |
| 95% | 0.97-1.51 | 1.02-1.95 | 0.77-1.43 | 0.64-1.43 |
| confidence | | | | |
| interval | | | | |
| p-value^{[a]} | 0.996 | 0.0406 | 0.7853 | 0.8419 |

^{[a]}Based on Cox regression model
ITT = intent-to-treat;
LDH = lactate dehydrogenase;
Not Ach. = not achieved;
ULN = upper limit of normal

These data demonstrate that a subject having a low level of LDH (equal to or less than the ULN) should be selected for treatment with elesclomol, and a subject having a high level of LDH (equal to or less than the ULN) should be selected against for treatment with elesclomol. These data further demonstrate that a subject having a low level of LDH (equal to or less than the ULN) is likely to benefit from treatment with elesclomol, and a subject having a high level of LDH (equal to or less than the ULN) is less likely or unlikely to benefit from treatment with elesclomol.

### Example 8

### Characterization of Treatment Outcomes to Demonstrate Improved Efficacy of Elesclomol in Subjects with Lung Cancer or Metastatic Melanoma with a Low Level of LDH

Clinical studies have been performed to demonstrate the efficacy of elesclomol in the treatment of lung cancer and melanoma. Clinical studies using elesclomol for the treatment of prostate cancer, solid tumors, recurrent or persistent ovarian epithelial cancer, fallopian tube cancer, and primary peritoneal cancer have also been planned or performed.

A chart review is performed to determine if levels of one or more hypoxic markers, particularly LDH, were analyzed for the subjects prior to, and optionally during treatment with elesclomol. If no information is available regarding the levels of hypoxic markers, serum samples retained from the study subjects are analyzed for LDH level and outcomes are analyzed in view of the LDH level.

Preliminarily, subjects within each of the groups, or at least the groups in which subjects were treated with elesclomol, are divided into high and low LDH level based on the upper limit of normal (ULN) for the site where the testing is done. A value equal to or less than the ULN is considered as low. Values greater than the ULN are considered high. Alternatively, low LDH can be considered as levels up to and including 0.8 ULN with high LDH being considered all values above 0.8 ULN. Alternatively, low LDH can be considered as levels up to and including 1.2 or 1.5 ULN with high LDH being considered all values above 1.2 or 1.5 ULN, respectively. It may be possible to further stratify the high and low ULN groups to provide further predictive power of the LDH level in predicting the response of a subject to treatment with elesclomol, e.g., assigning those with an LDH level of 1 to <2 times, or 1 to <3 times, etc. the ULN as having an intermediate or slightly elevated LDH level. Other cut-off values such as those provided in the instant application can also be selected. Statistical methods can be used to select appropriate cut-off levels. The outcome of the analysis is used to determine subjects who will most likely benefit from treatment with elesclomol. The outcome of the analysis is further used to select treatment regimens for subjects including or not including elesclomol based on the ULN level. Subjects with a low level of LDH are selected for treatment with elesclomol. Subjects with a high level of LDH are selected against for treatment with elesclomol.

### Example 9

### Trial to Demonstrate Improved Efficacy of Elesclomol in Subjects with Liver or Renal Cancers with a Low Level of LDH

Subjects are identified as having one of lung cancer or melanoma. A subject is selected as being candidate for treatment with elesclomol based on appropriate inclusion or exclusion criteria. Routine assessments are made prior to treatment to characterize the disease state of the subject including, but not limited to, imaging studies, hematological studies, and physical examination. Additionally, coded serum sample from the subject is tested to determine the LDH level. The results from the LDH level determination are not matched to the subject until the end of the treatment period. However, samples can be tested to allow sufficient numbers of subjects with low and high LDH levels to be recruited to provide sufficient power to the study.

Subjects are treated with at least one regimen including elesclomol, either alone or in combination with other agents. Depending on the number of subjects available and the scope of the trial, the two regimens can be compared, or all subjects can be administered a single regimen. At predetermined regular or irregular intervals, subjects are assessed for specific outcomes including, but not limited to, overall survival, progression free survival, time to progression, and adverse events. Treatment is continued for as long as the subject responds positively to treatment with the assigned regimen and there are no limiting adverse events. However, an arbitrary treatment window can be selected to allow for conclusion of the trial.

Upon conclusion of the trial, the results from the LDH level analysis are unblinded and matched to the subjects. As specific methods of testing are available, the amount of LDH is scored as being low or high based on the upper limit of normal (ULN) for the site where the testing is done. A value equal to or less than the ULN is considered as low. A value greater than the ULN is considered to be high. Alternatively, low LDH can be considered as levels up to and including 0.8 ULN with high LDH being considered all values above 0.8 ULN. Alternatively, low LDH can be considered as levels up to and including 1.2 or 1.5 ULN with high LDH being considered all values above 1.2 or 1.5 ULN, respectively. It may be possible to further stratify the high and low ULN groups to provide further predictive power of the LDH level in predicting the response of a subject to treatment with elesclomol, e.g., assigning those with an LDH level of 1 to <2 times, or 1 to <3 times, etc. the ULN as having an intermediate or slightly elevated LDH level. Other cut-off values such as those provided in the instant application can also be selected. Statistical methods can be used to select appropriate cut-off levels. The outcome of the analysis is used to determine subjects who will most likely benefit from treatment with elesclomol. The outcome of the analysis is further used to select treatment regimens for subjects including or not including elesclomol based on the ULN level. Subjects with a low level of LDH are selected for treatment with elesclomol. Subjects with a low level of LDH are selected against for treatment with elesclomol.

### Example 10

### Trial to Demonstrate Improved Efficacy of Elesclomol in Subjects with Leukemia with a Low Level of LDH

Subjects are identified as having one of relapsed or refractory acute myeloid leukemia (AML). A subject is selected as being candidate for treatment with elesclomol based on appropriate inclusion or exclusion criteria. Routine assessments are made prior to treatment to characterize the disease state of the subject including, but not limited to, imaging studies, hematological studies, and physical examination. Additionally, coded serum sample from the subject is tested to determine the LDH level.

This trial is expected to be sufficiently powered with the enrollment of 36 patients with relapsed or refractory AML and total baseline serum LDH level less-than or equal to 0.8 upper limit of normal (ULN). Patients are treated with elesclomol sodium on a once-weekly schedule at a starting dose of 200 mg/m², with dose escalation planned based on safety and tolerability. The primary endpoints are to characterize the safety and tolerability of elesclomol sodium and to determine the pharmacokinetics of elesclomol and its metabolites in this patient population. Secondary endpoints include assessing the activity of elesclomol as a monotherapy in the treatment of AML. Subjects with low levels of hypoxia are found to respond better to treatment with elesclomol than historical control populations with undetermined levels of LDH or high (i.e., greater than 0.8 ULN) levels of LDH.

### Example 11

### Method of Evaluating Activity Levels of LDH Isoforms in Samples

Human tumor cell lines HCT116 (ATCC #CRL-247; Schroy P C, et al. Cancer 76: 201-209, 1995) and 786-0 (ATCC #CRL-1932; Williams R D, et al. In Vitro 12: 623-627, 1976), were obtained from the American Type Culture Collection (Manassus, Va., USA) were cultured using routine methods until a sufficient number of cells were obtained for implantation. Studies were conducted on animals between 7 and 12 weeks of age at implantation. To implant HCT116 tumor cells into nude mice, the cells were trypsinized, washed in PBS and resuspended at a concentration of 75×10⁶ cells/ml in McCoy's modified medium with 50% of BD Matrigel® Basement Membrane Matrix (BD Biosciences®, Bedford, Mass., USA). To implant 786-O tumor cells into nude mice, the cells were trypsinized as above, washed in PBS and resuspended at a concentration of 75×10⁶ cells/ml in RPMI 1640 medium with 50% of BD Matrigel® Basement Membrane Matrix. Using a 27 gauge needle and 1 cc syringe, 0.1 ml of the cell suspension was injected into the corpus adiposum of nude mice. The corpus adiposum is a fat body located in the ventral abdominal vicera in the right quadrant of the abdomen at the juncture of the os coxae (pelvic bone) and the os femoris (femur).

The location permits palpation and measurement of the tumors using external calipers. Tumor volumes (V) were calculated by caliper measurement of the width (W), length (L) and thickness (T) of tumors using the following formula: V=0.5236×(L×W×T). Animals were randomized into treatment groups so that the average tumor volumes of each group were similar at the start of dosing.

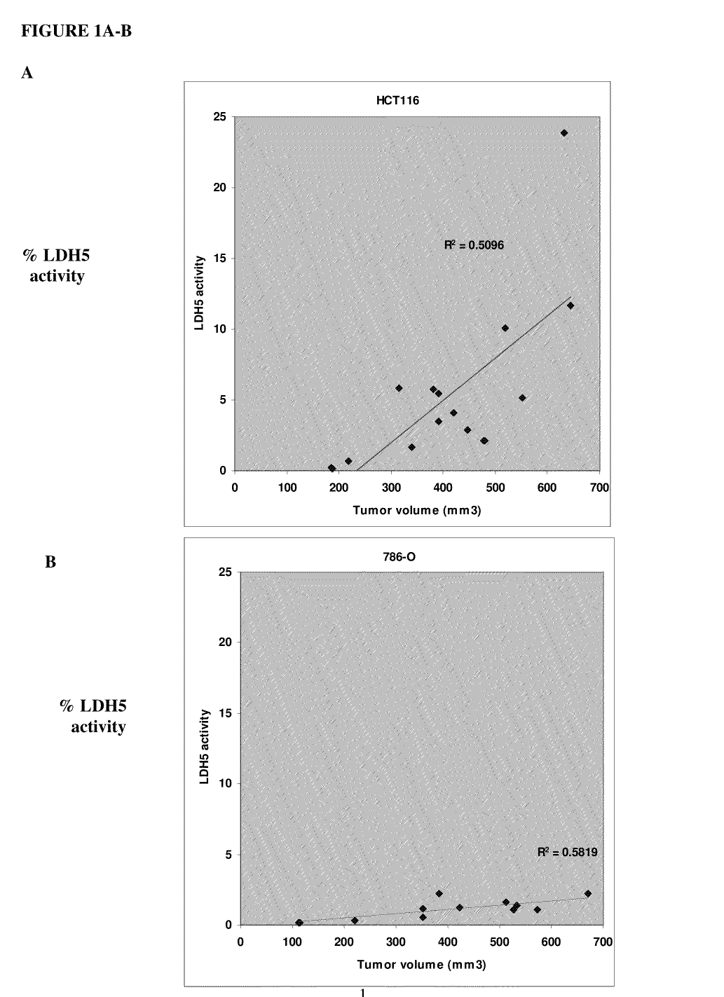

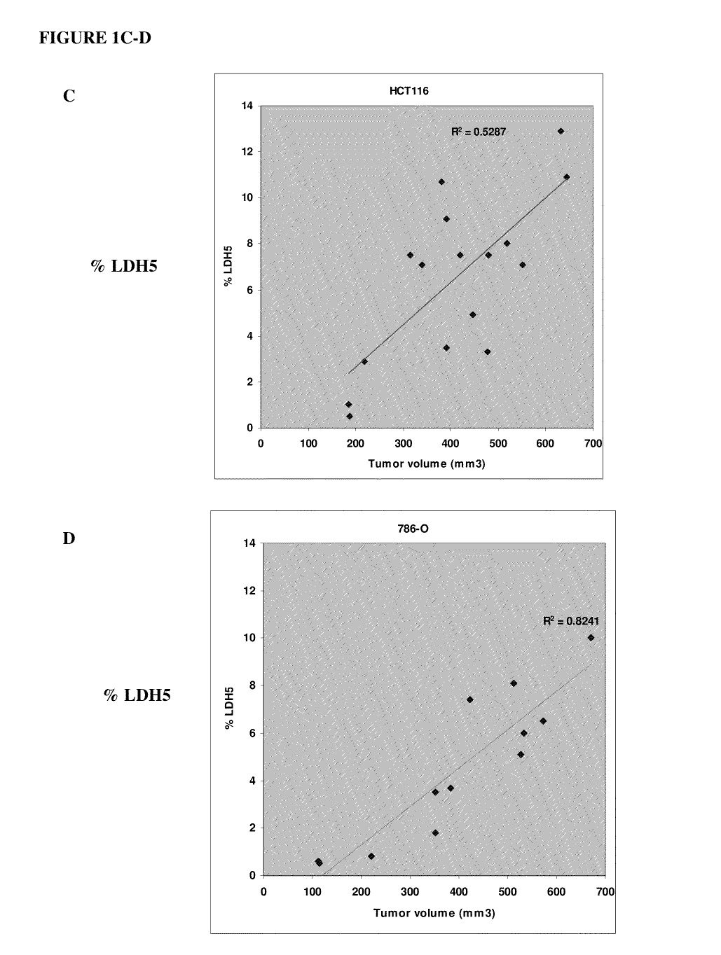

Blood was collected from the tumor bearing mice at appropriate time points, serum was prepared, and the serum frozen for later analysis. On the same days as blood collection, tumor volumes (V) were calculated by caliper measurement of the width (W), length (L) and thickness (T) of tumors using the following formula: V=0.5236×(L×W×T). After collection of the serum samples was completed, serum samples were resolved by gel electrophoresis. Following electrophoresis, the bands for the five isoenzymes were visualized by an enzymatic reaction using an in gel assay. Lactate, nicotinamide adenine dinucleotide (NAD+), nitroblue tetrazolium (NBT), and phenazine methosulphate (PMS) were added to assess LDH activity. LDH converts lactate to pyruvate and reduces NAD+ to NADH. The hydrogens from NADH are transferred by PMS to NBT reducing it to a purple formazan dye. The percentage of each LDH isoenzyme activity as well as the relative amount of LDH5 was determined by densitometry (Beckman Appraise densitometer, Beckman Coulter Inc. or Sebia (GELSCAN, Sebia Inc). The percent of LDH5 protein and LDH5 activity relative to the total LDH present (i.e., the amount of LDH5, LDH5, LDH3, LDH2, and LDH1 combined) was calculated and graphed against tumor volume. The results are shown in FIGS. 1A-D.

FIGS. 1A and 1B show the amount of LDH5 activity as a percent of total LDH activity as determined by the in gel assay. As shown, the HCT116 tumors had a substantially greater percent to LDH5 activity relative to total LDH activity as compared to the 7860 tumors. FIGS. 1C and 1D demonstrate that despite the difference in the relative activity of LDH5 that is observed, the amount of LDH5 protein present relative to total LDH is about the same for both tumor types.

### EQUIVALENTS

Those skilled in the art will recognize, or be able to ascertain using no more that routine experimentation, many equivalents to the specific embodiments of the invention described herein. Such equivalents are intended to be encompassed by the following claims.

### INCORPORATION BY REFERENCE

The contents of all references, patents, pending patent applications and published patents, cited throughout this application are hereby expressly incorporated by reference.

1. A method of treating a subject having cancer comprising:
administering elesclomol to the subject, wherein the cancer comprises a tumor with a low level of hypoxia.

2. The method of claim 1, wherein the level of hypoxia in the tumor is determined in a subject sample.

3. The method of claim 1, wherein the level of hypoxia in the tumor is determined by detecting the activity level or expression level of one or more hypoxia modulated polypeptides.

4. The method of claim 3, wherein the activity level or expression level of the one or more hypoxia modulated polypeptides are down regulated in the sample.

5. The method of claim 1, wherein the level of hypoxia is determined by detecting the activity level or expression level of one or more hypoxia modulated polypeptides or using detection methods selected from the group consisting of detection of activity or expression of at least one isoform or subunit of lactate dehydrogenase (LDH), at least one isoform or subunit of hypoxia inducible factor (HIF), at least one pro-angiogenic form of vascular endothelial growth factor (VEGF), phosphorylated VEGF receptor (pKDR) 1, 2, and 3; neurolipin 1 (NRP-1), pyruvate dehydrokinase (PDH-K), ornithine decarboxylase (ODC), glucose transporter-1 (GLUT-1), glucose transporter-2 (GLUT-2), tumor size, blood flow, EF5 binding, pimonidazole binding, PET scan, and probe detection of hypoxia level.

6. The method of claim 5, wherein the isoform or subunit of LDH comprises one or more selected from the group consisting of, LDH5, LDH4, LDH3, LDH2, LDH1, LDHA and LDHB; or any combination thereof including total LDH.

7. The method of claim 1, wherein detection of a low level of activity or expression of at least one LDH isoform or subunit comprises detection of an LDH activity or expression level of an LDH selected from the group consisting of total LDH, LDH5, LDH4, LDH5 plus LDH4, LDH5 plus LDH4 plus LDH3, and LDHA, wherein the activity level or expression level is 0.8 ULN or less.

8. The method of claim 1, wherein detection of a low level of activity or expression of at least one LDH isoform or subunit comprises detection of an LDH activity or expression level of an LDH selected from the group consisting of total LDH, LDH5, LDH4, LDH5 plus LDH4, LDH5 plus LDH4 plus LDH3, and LDHA, wherein the activity level or expression level is 1.0 ULN or less.

9. The method of claim 1, wherein a low level of hypoxia comprises a ratio or a normalized ratio of 1.0 or less of the ULN, wherein the ratio or normalized ratio is selected from the group consisting of the LDHA to LDHB, LDH5 or LDH4 to LDH1, LDH5 or LDH4 to total LDH, LDH5 and LDH4 to LDH1, LDH5 and LDH4 to total LDH, LDH5, LDH4, and LDH3 to LDH1, and LDH5, LDH4, and LDH3 to total LDH.

10. The method of claim 1, wherein the subject was previously treated with another chemotherapeutic agent.

11. The method of claim 1, further comprising identifying the subject as having a tumor with a low level of hypoxia.

12. A method for identifying a subject for treatment with elesclomol, comprising:
determining the level of hypoxia in a tumor from the subject, wherein a low level of hypoxia in the sample indicates the subject is likely to respond to therapy with elesclomol.

13. The method of claim 12, wherein a subject having a low level of hypoxia in the tumor is not likely to respond to therapy with elesclomol.

14-15. (canceled)

16. The method of claim 15, wherein the activity level or expression level of the one or more hypoxia modulated polypeptides are down regulated in the sample.

17. The method of claim 12, wherein the level of hypoxia is determined by detecting the activity level or expression level of one or more hypoxia modulated polypeptides or using detection methods selected from the group consisting of detection of activity or expression of at least one isoform or subunit of lactate dehydrogenase (LDH), at least one isoform or subunit of hypoxia inducible factor (HIF), at least one pro-angiogenic form of vascular endothelial growth factor (VEGF), phosphorylated VEGF receptor (pKDR) 1, 2, and 3; neurolipin 1 (NRP-1), pyruvate dehydrokinase (PDH-K), ornithine decarboxylase (ODC), glucose transporter-1 (GLUT-1), glucose transporter-2 (GLUT-2), tumor size, blood flow, EF5 binding, pimonidazole binding, PET scan, and probe detection of hypoxia level.

18-23. (canceled)

24. A kit for selecting a therapeutic regimen including elesclomol for the treatment of cancer comprising:
at least one reagent for determining the level of hypoxia of in a subject sample; wherein the level of hypoxia is used to select the treatment regimen including elesclomol.

25. The kit of claim 24, wherein a low level of hypoxia is indicative that a therapeutic regimen with elesclomol should be selected.

26-27. (canceled)

28. The kit of claim 24, wherein the level of hypoxia is determined by detecting an activity level or an expression level of one or more hypoxia modulated peptides.

29. The kit of claim 28, wherein the activity level or expression level of the one or more hypoxia modulated polypeptides are down regulated in the sample.

30. The kit of claim 24, wherein the level of hypoxia is determined by detecting the activity level or expression level of one or more hypoxia modulated polypeptides or using detection methods selected from the group consisting of detection of activity or expression of at least one isoform or subunit of lactate dehydrogenase (LDH), at least one isoform or subunit of hypoxia inducible factor (HIF), at least one pro-angiogenic form of vascular endothelial growth factor (VEGF), phosphorylated VEGF receptor (pKDR) 1, 2, and 3; neurolipin 1 (NRP-1), pyruvate dehydrokinase (PDH-K), ornithine decarboxylase (ODC), glucose transporter-1 (GLUT-1), glucose transporter-2 (GLUT-2), tumor size, blood flow, EF5 binding, pimonidazole binding, PET scan, and probe detection of hypoxia level.

30-46. (canceled)
